(19) United States
(12) Patent Application Publication
(10) Pub. No.: US 2003/0097059 A1
(43) Pub. Date: May 22, 2003

(54) Magnetic resonance spectroscopy to identify and classify microorganisms

(76) Inventors: Tania C. Sorrell, Riverview (AU); Carolyn E. Mountford, East Ryde (AU); Uwe Himmelreich, Mays Hill (AU); Roger Bourne, Marrickville (AU); Rajmund L. Somorjai, Headingly (CA)

Correspondence Address: Cooper & Dunham LLP, 1185 Avenue of the Americas, New York, NY, 10036, US

(21) Appl. No.: 10/081,838

(22) Filed: Feb. 21, 2002

Related U.S. Application Data
(60) Provisional application No. 60/270,367, filed on Feb. 21, 2001.

(51) Int. Cl. A61B 5/05

(52) U.S. Cl. 600/420

(57) ABSTRACT

A statistical classifier identifies microorganisms, such as bacteria and fungi, using magnetic resonance spectroscopy, with multivariate analysis.

The bacteria may include species within Staphylococcus, Enterococcus and Streptococcus. The fungi may include pathogenic yeasts including species with Candida and Cryptococcus.

### CROSS REFERENCE TO RELATED APPLICATIONS

This application claims priority on U.S. provisional application Serial No. 60/270,367, filed Feb. 21, 2001.

### BACKGROUND OF THE INVENTION

The present invention relates to identifying and classifying microorganisms, such as bacteria and fungi, using magnetic resonance spectroscopy, with multivariate analysis.

Throughout this application, various publications are referenced to within parentheses. Disclosures of these publications in their entireties are hereby incorporated by reference into this application to more fully describe the state of the art to which this invention pertains. Full bibliographic citations for these references may be found at the end of this application, preceding the claims.

Microbial taxonomic classification of micro-organisms involves the grouping of those with like characteristics, based on detection of multiple metabolities/compounds or analysis of genetic material (DNA), from microbial cells. “Gene trees” derived from sequences of the so-called ancestral ribosomal DNA gene, can distinguish between and within all living organisms down to species and, sometimes, individual strain level. Numerical algorithms and “trees” can also be constructed from profiles of microbial metabolities, provided the conditions of culture have been carefully standardised. In the medical setting, identification of microbial pathogens allows the clinician to predict and initiate appropriate therapy and to provide prognostic information to patients.

Sites of infected tissue are composites of microbial cells, host immune cells and usually, cells of the organ or tissue where the infection is localized. Pathological diagnosis traditionally is time-consuming and labour-intensive, being reliant on histopathological examination and microbial identification by morphology and culture, or, sometimes, other methods.

In both clinical and industrial laboratories methods for identification of microorganisms have historically been based on multiple phenotypic characters, including morphological features and a range of biochemical reactions. These tests are often time consuming and/or relatively expensive in their application and some are imprecise. Recently, alternative methods have been investigated in an attempt to develop a single, rapid method for characterization and identification of micro-organisms. These have included Fourier transform infrared spectroscopy (FTIRS) (11) (14), pyrolysis mass spectrometry (PyMS) (12), electrospray ionization mass spectrometry (EIMS)(7), UV resonance Raman spectroscopy (UVRRS) (15), and protein electrophoresis (16). While reports of these techniques suggest the possibility of rapid and reliable identification of some groups of microorganisms, most have been tested with small data sets. With the exception of FTIRS they are destructive techniques which analyze cellular decomposition products. All have the limitation that they do not directly yield information about the biochemistry of the intact viable organism. In contrast, magnetic resonance spectroscopy (MRS) of viable cells can provide information on a large range of metabolites. Biological applications of MRS most commonly exploit the non-invasive nature of the technique to study aspects of cellular biochemistry in living systems (6). However, not all applications of MRS require or include identification of the metabolites contributing to the MR spectrum. Pattern recognition techniques, which detect gross spectral characteristics associated with a-priori defined classes (such as pathological conditions), have been successfully applied to MRS of both tissues and body fluids. Accurate and reliable classifiers based on multivariate analyses of ¹H MR spectroscopic data, have been developed and validated for objective diagnosis of thyroid (21), ovarian (23), prostate (9), breast (13), and brain tumours (20). In some pathologies MRS is able to detect malignancy before morphological manifestations are visible by light microscopy (17).

Cryptococcosis, caused by C. neoformans, is a potentially life-threatening mycosis of immunocompromised and healthy hosts. C. neoformans is the commonest cause of fungal meningitis (1) and circumscribed lesions (cryptococcomas) can occur in both lung and brain (2, 3). Cerebral cryptococcomas have been reported in up to fourteen percent of Australian patients presenting with cryptococcosis, depending on host immune status at diagnosis (4). Brian lesions are usually diagnosed after C. neoformans has been identified in tissue or fluids obtained from other body sites, or in cerebrospinal fluid (CSF).

Brain biopsy is required for diagnosis when lesions are confined to the brain (3) or in the absence of other diagnostic material, as the pathology of infective lesions cannot be reliably distinguished by modalities such as computed tomography (5) or magnetic resonance imaging (MRI) (6).

Proton magnetic resonance spectroscopy (¹H MRS) has been applied to tumours, stroke and bacterial infections (7-14). In vivo MRS of the brain was developed and comprehensively tested for the diagnosis of human tumours (7, 9, 13) based on initial ex vivo and in vivo studies in animal models (15). MRS has identified tumour pathology in human biopsies with a very high sensitivity and specificity (16-20).

MR-visible compounds from micro-organisms and/or cells recruited during the host immune response may give rise to diagnostic and prognostic markers. Extracellular carbohydrates and other products of C. neoformans have been identified in CSF from patients with cryptococcal meningitis (21). Cells of C. neoformans are distinguished from those of other invasive fungal pathogens by an external polysaccharide capsule, which comprises a high percentage of the biomass in cryptococcomas. The purified capsular material has been studied by IH and ¹³C MRS (22). More recent studies have identified extracellular products of C. neoformans cultured in vitro using MRS (23).

Pathogenic bacteria and fungi are normally identified and classified on the basis of their cellular morphology and biochemistry. Traditional methods are usually time-consuming, as several physiological tests are required for unequivocal identification. Where only minor pheno- and chemotypic differences exist, as for some species of the yeast genus Candida (64), such tests may be difficult or even fail to be definitive. Genotypic methods are more accurate but labor intensive and expensive.

### SUMMARY OF THE INVENTION

It is an object of the present invention to provide a statistical classifier for enabling the identificaitn, preferably down to the species group or species level, of various microorganisms. As used herein, the term “microorganism” means any microscopic organism (i.e., any unicellular or multicellular living entity) including bacteria, fungi, parasites, viruses, protozoa and algae.

According to the present invention a one-dimensional ¹H MR spectrum of a microorganism such as a bacterial cell suspension provides an overview of hydrogen-containing compounds. Consequently, the ¹H MR spectrum is more representative of the physiology of the cell (metabolite pools) than its structure (comprising immobile components such as the cell wall). While many different bacterial groups may express and utilize essentially identical metabolic pathways, differing levels of enzyme expression and activity in different groups could give rise to distinctly different levels of particular metabolities when dissimilar groups are grown in similar environments. It was, therefore, proposed that significantly different metabolite pool sizes could be detected as differences between the ¹H MR spectra of the different bacterial groups. This was suggested in a previous study comparing selected bacterial ¹H MR spectra (5), however the small number of isolates examined and the qualitative identification methods described in that study did not permit automation or quantitative comparison of the species groups.

According to the present invention, the use of linear discriminant analysis (LDA) on cultures of different microorganism isolates, such as different species of bacteria, enabled reliable automated identifications of the bacteria species to be made on the basis of their ¹H MR spectra.

According to the present invention, a new fingerprinting technique is provided for identification of microorganisms, bacteria, by combining proton magnetic resonance spectroscopy (¹H MRS) with multivariate statistical analysis. This has resulted in an objective identification strategy for common clinical isolates belonging to the bacterial species Staphylococcus aureus, Staphylococcus epidermidis, Enterococcus faecalis, Streptococcus pneumoniae, Streptococcus pyogenes, Streptococcus agalactiae, and the Streptococcus milleri group. A total of 312 cultures of 104 different isolates were examined using ¹H MRS. An optimized classifier was developed using a bootstrapping process and LDA to provide objective classification of the spectra. Identification of isolates was based on classification of spectra from duplicate cultures and achieved 94% agreement with conventional methods of identification. Less than 1% of isolates were identified incorrectly. Identification of the remaining 5% of isolates was defined as indeterminate. A small number of isolates of Enterococcus casseliflavus and E. gallinarum were examined and could be distinguished from E. faecalis, with 96% agreement with conventional identification methods.

According to the present invention, MRS is able to identify metabolites that identify and distinguish between micro-organisms. When combined with LDA, MRS can identify species of pathogenic bacteria that belong to different genera(65). According to the present invention, MRS with LDA enabled identification of pathogenic micro-organisms that are taxonomically closer related then shown by Bourne et al(⁶³). For example, five pathogenic Candida species as well as the two varieties the pathogenic yeast species Cryptococcus neoformans (var. gattii and var. neoformans) can be identified according to the invention.

According to the present invention, a method for obtaining a statistical classifier for classifying microorganisms of unknown species into known species is provided, comprising (a) obtaining a plurality of magnetic resonance spectra of each of a plurality of different species of microorganisms whose species is known, (b) locating a plurality of maximally discriminatory subregions in the magnetic resonance spectra obtained, and (c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, which coefficients and classifier spectra can be used to determine the species of microorganisms whose species are unknown.

According to another aspect of the invention, a method for determining the species of a microorganism of unknown species is provided, comprising obtaining magnetic resonance spectra of the microorganism of unknown species, and comparing the spectra obtained with a species classifier, said classifier having been obtained by (a) obtaining a plurality of magnetic resonance spectra of each of a plurality of different species of microorganisms whose species is known; (b) locating a plurality of maximally discriminatory subregions in the magnetic resonance spectra obtained, and (c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, and selecting, as the species of the unknown species of microorganism, the microorganism whose spectra has the closest match to the spectra of the unknown microorganism species.

According to the present invention, a method for obtaining a statistical classifier for classifying microorganisms of unknown species into known species is provided, comprising (a) obtaining a plurality of magnetic resonance spectra of each of a plurality of different species of microorganisms whose species is known, (b) locating a plurality of maximally discriminatory subregions in the magnetic resonance spectra obtained, and (c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, which coefficients and classifier spectra can be used to determine the species of microorganisms whose species are unknown.

### DESCRIPTION OF THE DRAWINGS

FIG. 1A shows representative ¹H MR spectra: of E. faecalis, S. milleri, S. pneumoniae and S. pyogenes isolates. Refer to Table 3 for the identity of the major metabolites contributing to the spectra in each integration region.

FIG. 1B shows representative ¹H MR spectra of: S. epidermidis, S. aureus and S. agalactiae isolates. The intense betaine peaks in the spectra of S. aureus and S. epidermidis and the GPC peak of S. agalactiae have been truncated to show details of the less intense peaks. The relative intensities of the betaine and GPC peaks can be seen in FIG. 2. Refer to Table 3 for the identity of the major metabolites contributing to the spectra in each integration region.

FIGS. 2A and 2B show Range of measured integral intensities for each species group, the bars showing mean±SD.

FIG. 3 shows: iD ¹H MR spectra from in vitro cell cultures: A) Crytococcus neoformans, B) Candida albicans, C) Aspergillus fumigatus, D) Saccharomyces cerevisiae and E) C6 cell line. Identification of the resonances: AA, amino acids, ac, acetate; CH, nonspecific carbohydrate resonances; lip, lipids; NCH, contributions from creatine, GABA, lys residues; N(CH₃)₃, contributions from choline containing compounds (chol, PC, GPC), betaine and tau; tre, a,a-trehalose. Note the prominent trehalose resonances in the spectrum from C. neoformans, which are not distinguishable in the other spectra;

FIG. 4 shows 1D and 2D COSY MR spectra from cerebral rat tissue samples: (a) control brain tissue, (b) tissue infected with C. neoformans, and (c) tissue with glioma. Identification of the resonances: A-G, triglyceride resonances (32), AA, amino acid residues; ac, acetate; ala, alanine; chol, choline; EA, ethanolamine; eth, ethanol; GABA, y-amino butanoic acid; glu/ gln, glutamate/ glutamine; GPC, glycerol-phosphocholine; h-tau, hypo-taurine; ile, isoleucine; lac, lactate; leu, leucine; lip, lipid; lys, lysine; mI, myo-inositol; NAA, N-acetyl aspartate; NCHₙ, contributions from creatine, phospho-creatine, GABA, lysine; N(CH₃)₃, contributions from choline containing compounds (choline, PC, gPC), betaine and taurine PC, phosphocholine; PE, phosphoethanolamine; tau, taurine; thr, threonine; tre, -trehalose; val, valine. The listed amino acids refer to amino acid residues and not necessarily to the respective free amino acids; and

FIG. 5 is a block diagram of a system according to the invention.

### DESCRIPTION OF THE PREFERRED EMBODIMENTS

According to the present invention, a method for obtaining a statistical classifier for classifying microorganisms of unknown species into known species is provided, comprising (a) obtaining a plurality of magnetic resonance spectra of each of a plurality of different species of microorganisms whose species is known, (b) locating a plurality of maximally discriminatory subregions in the magnetic resonance spectra obtained, and (c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, which coefficients and classifier spectra can be used to determine the species of microorganisms whose species are unknown.

The method preferably further comprises the step of repeating step (c) a plurality of times, each time selecting as the first portion of the spectra a different portion of the spectra from the species, to obtain a different set of optimized linear discriminant analysis coefficients for the species, and obtaining a weighted average of the linear discriminant analysis coefficients to obtain final classifier spectra. The step of cross-validating the spectra preferably comprises cross validating the spectra by randomly selecting about half of the spectra. The step of repeating step (c) a plurality of times preferably comprises repeating step (c) about 1000 times.

The method preferably includes the steps of obtaining a plurality of classifier spectra independently, and aggregating the results of the independent classifiers to obtain a consensus diagnosis.

The microorganisms may include bacteria, including the species of Staphylococcus aureus, Staphylococcus epidermidis, Enterococcus faecalis, Enterococcus casseliflavus, Enterococcus gallinarum, Streptococcus pneumoniae, Streptococcus pyogeries and Streptococcus agalachae.

The microorganisms may include fungi, including pathogenic yeasts such as Candida albicans, Candida parapsilosis, Candida tropicalis, Candida krusei, and Candida glabrata, and Cryptococcus varieties of neoformans and gattli. The microorganisms may include cultured bacterial infections, and/or specimens from a mammal containing bacterial infections.

The plurality of magnetic resonance spectra of each different species is preferably at least 10, and may be at least 30.

According to another aspect of the invention, a method for determining the species of a microorganism of unknown species is provided, comprising obtaining magnetic resonance spectra of the microorganism of unknown species, and comparing the spectra obtained with a species classifier, said classifier having been obtained by (a) obtaining a plurality of magnetic resonance spectra of each of a plurality of different species of microorganisms whose species is known; (b) locating a plurality of maximally discriminatory subregions in the magnetic resonance spectra obtained, and (c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, and selecting, as the species of the unknown species of microorganism, the microorganism whose spectra has the closest match to the spectra of the unknown microorganism species.

The method preferably further comprises the step of repeating step (c) a plurality of times, each time selecting as the first portion of the spectra a different portion of the spectra from the species, to obtain a different set of optimized linear discriminant analysis coefficients for the species, and obtaining a weighted average of the linear discriminant analysis coefficients to obtain final classifier spectra. The step of cross-validating the spectra preferably comprises cross validating the spectra by randomly selecting about half of the spectra. The step of repeating step (c) a plurality of times comprises repeating step (c) about 1000 times. The method preferably includes the steps of obtaining a plurality of classifier spectra independently, and aggregating the results of the independent classifiers to obtain a consensus diagnosis. The microorganisms may include bacteria, including the species of Staphylococcus aureus, Staphylococcus epidermidis, Enterococcus faecalis, Enterococcus casseliflavus, Enterococcus gallinarum, Streptococcus pneumoniae, Streptococcus pyogeries and Streptococcus agalachae.

The microorganisms may include fungi, including pathogenic yeasts such as Candida albicans, Candida parapsilosis, Candida tropicalis, Candida krusei, Candida glabrata, and Cryptococcus varieties of neoformans and gattli. The microorganisms may include cultured bacterial infections, and/or specimens from a mammal containing bacterial infections.

The plurality of magnetic resonance spectra of each different species is preferably at least 10, and may be at least 30.

According to another aspect of the invention, a statistical classifier for classifying microorganisms of unknown species into known species is provided, comprising (a) a spectrometer for obtaining a plurality of magnetic resonance spectra of each of a plurality of different species of microorganisms whose species is known, (b) a locator for locating a plurality of maximally discriminatory subregions in the magnetic resonance spectra obtained, and (c) a cross-validator for cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, which coefficients and classifier spectra can be used to determine the species of microorganisms whose species are unknown.

The cross-validator preferably repeats step (c) a plurality of times, each time selecting as the first portion of the spectra a different portion of the spectra from the species, to obtain a different set of optimized linear discriminant analysis coefficients for the species, and obtaining a weighted average of the linear discriminant analysis coefficients to obtain final classifier spectra. The cross-validator preferably cross validates the spectra by randomly selecting about half of the spectra. The classifier preferably repeats step (c) about 1000 times. The classifier preferably obtains a plurality of classifier spectra independently, and aggregates the results of the independent classifiers to obtain a consensus diagnosis.

The microorganisms may include bacteria, such as Staphylococcus aureus, Staphylococcus epidermidis, Enterococcus faecalis, Enterococcus casselifavus, Enterococcus gallinarum, Streptococcus pneumoniae, Streptococcus pyogeries, and Streptococcus agalachae.

The microorganisms may include fungi, including pathogenic yeasts such as Candida albicans, Candida parapsilosis, Candida tropicalis, Candida krusei, Candida glabrata, and Cryptococcus varieties of neoformans and gattli. The microorganisms may include cultured bacterial infections and/or specimens from a mammal containing bacterial infections.

The plurality of magnetic resonance spectra of each different species is preferably at least 10 and may be at least 30.

A number of examples will be described for identifying different types of microorganisms, in particular different bacteria and fungi. However, the present invention is not limited to the species of bacteria and fungi in the examples, and can be used for any microorganism capable of being identified through the method disclosed herein.

### EXAMPLE 1

### Bacteria Detection

1. Storage and Culture of Bacteria

Isolates were obtained from the collection of the Centre for Infectious Diseases and Microbiology (CIDM), Institute of Clinical Pathology and Medical Research, Sydney and the American Type Culture Collection, or were recent clinical isolates from the clinical identification laboratory of the CIDM Laboratory services. Stored isolates were suspended in 10% glycerol in nutrient broth at −70° C. Horse blood agar (HBA) was prepared by addition of sterile horse blood to autoclaved blood agar base (Oxoid (UK) or Amyl Media (Australia)). Isolates retrieved from storage were subcultured on to 5% horse blood agar and incubated in 5% CO₂ for 18-24 hours at 37° C. New isolates and isolates subcultured on HBA after storage were streaked onto duplicate HBA plates and incubated at 37° C. for 18-24 hours and then stored at ambient temperature (20-30° C.) for 3-9 hrs before spectroscopy.

To test for short-term method variability, examined duplicate cultures of all isolates were examined. To test for long-term culture and method variability a number of isolates we recultured up to seven times over an eight month period. Included in the analysis were spectra of 3 isolates of Enterococcus gallinarum and 3 isolates of E. casseliflavus which are closely related to E. casseliflavus which are closely related to E. faecalis (10) (Table 1). The number of distinct isolates examined from each species group, and the number of times the isolate was recultured and reexamined can be determined from Table 1.

2. Conventional Identification of Bacteria

Staphylococcus aureus was identified on the basis of positive coagulase (using rabbit or human plasma) and DNase tests. Staphylococcus epidermidis was identified using the API ID32 staph test (BioMerieux, France). Streptococcus and Enterococcus species were identified by conventional methods—optochin sensitivity (Streptococcus pneumoniae), salt tolerance and bile-esculin positivity (Enterococcus spp.), latex agglutination (Streptococcus agalactiae), and by API ID32 strep test (BioMerieux). All tests were carried out according to the manufacturers' instructions. In general, isolates were identified only once upon receipt in the microbiology laboratory and prior to storage. Some isolates retrieved from storage were reidentified by conventional tests.

3. ¹H MR Spectroscopy

Bacterial colonies (2-200 mg wet weight) were gently removed from the HBA plate with a plastic inoculating loop and suspended by vortex in 0.3 mL phosphate buffered saline (pH 7.2, room temperature) made up in D₂O (PSB/D₂O). For most cultures >80% of cells were scraped off the plate. In cases of heavy growth <10% of cells were harvested, usually from the first quadrant. The suspension was immediately transferred to a 5 mm susceptibility-matched MR sample tube (Shigemi, USA). ¹H MRS measurements were performed at 37° C. on a Bruker Avance 360 MHz MR spectrometer using ¹H/¹³C 5 mm probe head. 1D spectra were acquired with acquisition parameters as follows: frequency 360.13 MHz, pulse angle 900 (6-7 ms), repetition time is, 8k data points, 256 or 512 transients, spectral width 3600 Hz, total acquisition time 10 or 20 min. The field was locked to D₂O. Water suppression was effected by a selective excitation field gradient method (DPFGSE, (3)). Spectra of cells suspended in PBS/D₂O were stable for at least two hours at 37° C.

4. Signal Assignment

Two dimensional (2D) homo- and heteronuclear correlation spectra were acquired for at least two isolates per species to assign ID MR resonances to specific compounds. {¹H, ¹H} gradient COSY experiments were performed in magnitude-mode. Acquisition parameters were: sweep width in t₂ 3600 Hz, t₂ time domain 2K, 256 increments of 32 or 48 acquisition each, repetition time is. Sine-bell window functions were applied in the t₁ dimension, and Gaussian-Lorentzian window functions were applied in the t₂ dimension. Zero filling was used to expand the data matrix to 1 K in the t₁ dimension. TOCSY spectra with mixing times of 40 ms and 150 ms were acquired with 256 increments of 2K data points and 32 acquisitions (1). {¹H, ¹³C} one-bond shift correlation spectra were obtained in the ¹H detection mode using a gradient HSQC pulse sequence (24). The ¹H MR spectral width was 3600 Hz and the ¹³C MR spectral width was 15000 Hz. ¹³C MR decoupling during acquisition was achieved by GARP-1 (18). The evolution time (t₁) was incremented to obtain 400 FIDs, each of 40-64 acquisitions and consisting of 2K data points. The repetition time was 1 s. A sine-bell function was applied in the t₂ dimension and a Gaussian-Lorentzian function was applied in the t₁ dimension. Zero filling to 1 K was used in the t₁ dimension prior to Fourier transformation. {¹H, ¹³C} gradient Heteronuclear Multiple Bond Correlation spectra (HMBC) were acquired without proton decoupling using the same parameters as for the HSQC experiments except for a ¹³C MR spectral width of 20 kHz (24). One-bond and long-range correlation experiments were usually optimized for ¹J_{C,H} of 140 Hz and ⁿJ_{C,H} of 7 Hz respectively. 1D ¹H MR spectra were acquired before and after the 2D experiments to verify absence of metabolic changes.

5. Data Processing

Spectra were processed using Bruker XWINNMR spectrometer software. Zero filling was performed to extend the free induction decay data set to 16K. An exponential window function was applied before Fourier transformation yielding a line broadening of 1 Hz. Chemical shift calibration was performed by setting the center of the spectrum to 4.64 ppm (nominal position of the water resonance with respect to tetramethylsilane in PBS/D₂O at 37° C.). Spectra were manually phase corrected to achieve a linear and flat baseline.

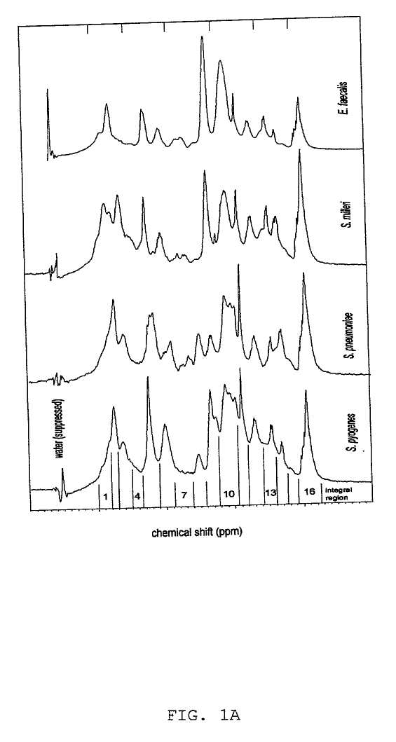

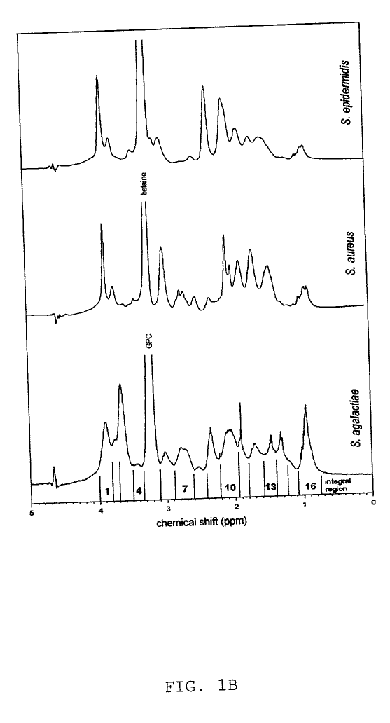

Sixteen contiguous fixed integration regions were subjectively chosen on the basis of major peaks present in the representative spectra (FIG. 1). The individual integrals were normalized to the total intensity of the 16 integrals between 4.0 and 0.75 ppm.

6. Linear Discriminant Analysis

The table of integrals was imported from Microsoft Excel into STATISTICA (StaSoft Pacific P/L, Australia) for LDA. Each of the first 15 of 16 chosen integral regions (see Results) formed one independent variable in the LDA (Standard method, 7 groups, Tolerance 0.01, a priori classification probability proportional to group size). The 16^{th} region was omitted because one region is redundant for discriminant analysis in a normalized data set. Classification functions and classification probabilities were calculated with STATISTICA.

7. Classification of Spectra and Identification of Isolates

The following definitions are used herein. The term classification refers to assignment of an individual spectrum from a bacterial culture to a species group. Identification refers to assignment of an isolate to a species group (on the basis of classification of two independent spectra derived from duplicate cultures of the isolate). Correct classification refers to assignment of a spectrum to the same species group as conventional classification with a percent classification probability >75%. Misclassification refers to assignment of a spectrum to a species group different from conventional classification with a percent classification probability >75%. Indeterminate classification refers to assignment of a spectrum to any species group with percent classification probability ≦75%. Correct identification refers to assignment of both spectra of duplicate cultures according to conventional identification and with average of percent classification probability >75%. Misidentification refers to assignment of both spectra of duplicate cultures to the same species group but different from conventional identification and with average of percent classification probability >75%. Indeterminate identification refers to assignment of spectra of duplicate cultures to different groups, or the same group with average classification probability <75%.

An optimized classifier for all 7 species groups was developed based on the Robust BootStrap (RBS) method of Somorjai et al. (19), (2). Starting with all 312 spectra, half the spectra were randomly selected from each species group and this training set was used to train the 7-group classifier (LDA). The resulting classifier was then used to validate the remaining spectra (the test set). This process was repeated B times (with replacement) and every time the optimized LDA coefficients were saved. The weighted average of these B sets of LDA coefficients produces the final classifier(B=1000). The weight for the mth set is Wₘ=KₘCₘ^{1/2}, m=1, . . . , B, where 0<Cₘ<1 is the crispness (defined as the fraction of test samples assigned to a class with percent probability 75%, and 0<Kₘ<1 is Cohen's chance-corrected measure of agreement (4), Kₘ=1 signifying perfect classification of a test set. The B values Wₘ used for the weights are those obtained not for the bootstrap training sets, but for the less optimistic test sets. The optimized classifier was then used to classify all 312 spectra. Classifier outcome is reported as a percent class probability.

For separate classification of Enterococcus spp. an optimized classifier was developed based on RBS of the 62 spectra of three Enterococcus spp. (B=200, LDA parameters as above).

The RBS classification software was written using STATISTICA, Microsoft EXCEL and Microsoft VISUAL BASIC for APPLICATIONS (VBA) and run on a Pentium-based personal computer.

8. Results

¹H MR Spectra.

Representative spectra of each of the 7 species groups and the 16 integration regions chosen for analysis, are shown in FIG. 1. Spectra of ATCC type strains are shown where available, otherwise a spectrum of an isolate close to the group centroid (based on integral intensities) of all spectra is shown. The most significant contributing metabolities identified for each integration region and used for the statistical analyses, are listed in Table 3.

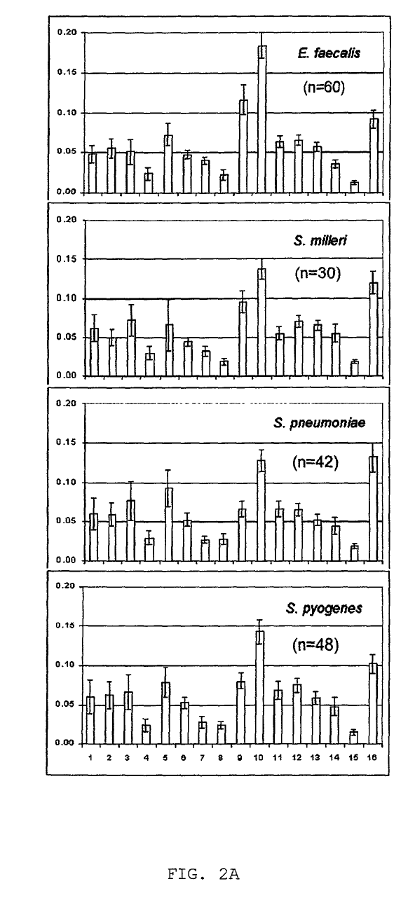

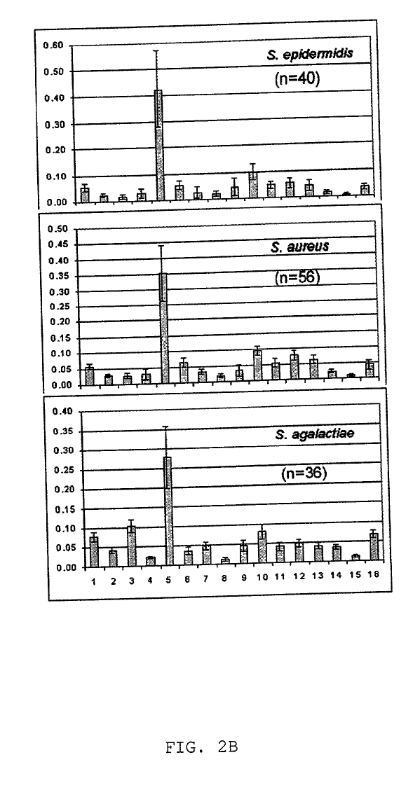

While it is not possible to show the range of spectral patterns found in the 30-60 spectra examined from each species group, FIG. 2 shows the range of normalized integral intensities (mean±SD) measured for each species group.

9. Classification of Spectra and Identification of Isolates

Results for classification of 312 spectra and identification of 104 isolates from the seven species groups based on the optimized classifier are shown in Table 1. A summary of results in terms of classification and identification performance is shown in Table 2. Less than 2% of spectra were misclassified and less than 1% of isolates misidentified. There were 13 spectra which had indeterminate classification. Of the 9 isolates which showed indeterminate identification 5 were correctly identified in subsequent or previous cultures at different dates (the remaining 4 isolates were not retained in culture storage and could not be retested).

The results of an attempt to classify the 14 spectra of three Enterococcus casseliflavus and three E. gallinarum isolates separately from E. faecalis are shown in Table 4. Notwithstanding the small number of isolates examined, these results suggest that it may also be possible to reliably identify E. casseliflavus and E. gallinarum separately from E. faecalis.

10. Reproducibility of Spectra

Independent analysis of spectra from concurrent, duplicate, cultures and of isolates retrieved repeatedly from storage over a 1-8 month period, confirmed that the classification method is robust and not affected by short or long-term procedural variability due to factors such as minor changes in culture conditions, number of organisms, or storage of isolates (see Table 1).

11. Discussion

a. ¹H MRS and Selection of Independent Variables for Multivariate Analysis.

Visible differences between the typical spectra of some species are readily observed, as seen in FIG. 1. However, differences between the spectra of species such as S. pyogenes and S. pneumoniae are not obvious by visual inspection and the only possibility of reliably distinguishing between such similar groups lies in a multivariate analysis of the data. The initial step in such an analysis is the extraction from the spectra, which are comprised of many thousands of data points, of a manageable set of independent variables in which any significant group differences are manifest. While sophisticated methods have been described for selection of optimally discriminating spectral regions (21) a simple division was chosen of all spectra into 16 contiguous regions visually selected on the basis of peaks present in the spectra illustrated in FIG. 1. The advantage of this procedure is that the resultant independent variables may be assigned a specific biochemical significance (i.e., an independent variable may be associated with a particular metabolite or group of metabolites) if the metabolites contributing to the signal in each integration region can be identified. Although Table 3 identifies some of the major metabolites contributing to the spectra in FIG. 1, the bacterial identification method applied here does not depend on identification or quantitation of the metabolites contributing to the MR signal. It is, however, important to note that the measured cellular characteristics on which the classification is based are substantially different from those detected during routine identification and also different from those measured by other whole organism fingerprinting techniques.

b. Classification and Identification Strategy

Classification based on LDA requires that a set of functions derived by LDA of a training set of data be used to classify a test set of data, which is preferably independent of the training set (cross-validation). The function of the training set is to describe, in terms of the n independent variables derived from the MR spectra, the region of n-dimensional data space occupied by each of the a priori defined groups. If the defined groups in the training set are well separated in data space, then the LDA will produce classification functions which assign every member of the training set to its a priori defined group. The region of data space associated with a particular group will increase with phenotype variation between the members of a particular species group, and also with procedural (environmental, biochemical and methodological) variation associated with repeated culture and classification of spectra of a specific member of a group. A training set comprising only a small number of randomly selected members of a particular group is thus unlikely to accurately represent the data space (phenotype range) occupied by all members of that species group. If the training set contains only a single measurement of each isolate member then it may also not account for procedural variability. Consequently, it is to be expected that some misclassifications will occur when a classification function based on a training subset of a group is used to classify group members which are not members of the training set.

For classifier robustness and reliability it is desirable that the number of spectra per species group in the training set be 5-10 times more than the number of independent variables (19). Such large data set appear to be rare, and are usually difficult to acquire, especially if the derived classifier is to be validated against a test set independent of the training set. The Robust BootStrap method attenuates this problem by allowing cross-validated classifier development with all of the available data (19).

The ease of preparation and examination of duplicate or even triplicate cultures of a particular clinical isolate, as used herein, has the advantage that a consensus identification of the isolate based on multiple independent analyses, is obtained. This feature of the isolate identification strategy has not been applied in other microbial whole organism fingerprinting studies (5, 8) in which, at best, only instrument duplicates were acquired. In a few cases the duplicates may be incorrectly classified as different species. Consequently, identification based on analysis of a single subculture of an isolate cannot be assigned the same confidence level as an identification based on classification of independent duplicate cultures. When using conventional methods, which report an identification probability based on analysis of a single culture of an isolate, it is common practice to reexamine isolates for which the identification probability <75%. Analysis is repeated until a single test returns an identification probability >75%. By this method it is possible that the average identification probability of all tests on an isolate will be<75% at the conclusion of testing. The method of always testing duplicate cultures and requiring that correct identification be based on an average probability >75% imposes a more rigorous and reliable identification constraint. In an applied (clinical laboratory) environment, in which testing of duplicates is made a routine procedure, disparity between the assigned group of duplicates should be interpreted as an indicator of procedural problems and such isolates should be retested or examined with supplementary techniques. Phenotypic variability within species groups was addressed by examination of at least 11 isolates from each species group. The general success of the classification method used indicates that between the species groups there are significant and consistent spectral differences which are larger than the typical range of variation within species due to procedure or phenotype.

C. Classification and Identification Results

The very small number of misclassifications of spectra could not be attributed to any specific steps of the method. Potential problems with reproducibility due to short- and long-term procedural variability (use of different batches of culture medium, storage of isolates, etc) were excluded by undertaking separate analysis of spectra from duplicate cultures of all isolates and reculture and reclassification of spectra of 25 isolates, at times up to 8 months after original culture and spectroscopy. The single instance of misidentification (S. pyogenes Lab. No. 221-2985) may have been the result of contamination. Previous and subsequent tests of the same isolate gave correct identification results. There are several characteristics of the method utilized herein which point to the robust nature of the identification.

Firstly, the growth conditions for the samples are not strictly controlled. For example, the precise constitution of the growth medium may vary from batch to batch (base media from two different manufacturers and multiple batches of horse blood were used). The size of the inoculum may vary from plate to plate. Growth of bacteria on an agar plate is inherently inhomogeneous, due to crowding and slow diffusion of oxygen and other nutrients through colonies and agar. Early experiments with triplicate cultures of all isolates demonstrated a lack of variation in spectra from cells grown on single batches of medium. Due to large variations between species in the amount of growth obtained overnight on HBA plates (growth of S. milleri was usually very poor) the wet weight of cells resuspended varied from 2-200 mg. As the MR signal is directly proportional to sample concentration, poor bacterial growth required only an extended number of transients to achieve adequate signal to noise ratio. The phase correction and integration steps of spectrum processing, as implemented, required some subjective operator input. These deficiencies in the method will introduce some extra variance into the data. They may be overcome by use of magnitude spectra and automated integration (23). Other whole organism fingerprinting techniques are reported to require strict control of growth media and repeated standardization with control cultures (12), (11).

A sufficient number of isolates in the S. milleri group were examined to attempt an MRS based assignment of the isolates to one of the three species within the S. milleri group (S. anginosus, S. constellatus, S. intermedius). However, the results demonstrate that the group is physiologically homogeneous relative to the diversity of the seven species groups examined. Similarly, the E. casseliflavus and E. gallinarum isolates examined are physiologically more similar to E. faecalis than to the Streptococcus and Staphylococcus species tested.

d. Choice of Growth Medium

In selecting the most appropriate medium for use in a clinical diagnostic or reference laboratory, a universal growth substrate and ease of sample preparation were of prime importance. Since HBA is a common medium in use in diagnostic microbiology laboratories and bacterial cells could be easily harvested directly from HBA plates without the need for washing, this growth medium was chosen as best satisfying the objectives.

There were major differences between spectra obtained herein and those published for S. aureus and E. faecalis grown on trypticase soy sheep blood agar (5). In the latter study, interpretation of spectral patterns was reportedly not affected by the choice of growth medium, possibly because spectral patterns were inspected visually and distinguished by peak positions rather than peak intensities. Growth on or in different media (HBA versus brain hear infusion broth) affected relative peak intensities due to changes in metabolite pool sizes, much more significantly than peak positions, which may be slightly affected by factors such as intracellular pH.

e. Clinical Application

Though based on a limited set of gram positive bacteria, the results suggest that ¹H MR spectroscopy of whole cells is of comparable precision and accuracy to established, automated, methods of species identification. These include common laboratory systems such as VITEK (22). The non-destructive nature of the method enables retention of viable organisums post-analysis for subsequent checking of contamination or methodological errors.

The use of more sophisticated pattern recognition methods than those used herein (see(19)) may further improve discrimination and allow separate classification within the species groups, albeit at the possible expense of easily interpreted biochemical information. For an application dedicated to identification rather than characterization, this would be an acceptable compromise. The extreme ease of sample preparation, biochemically informative results, rapid automated identification, and the robust nature of the method are attractive for clinical and industrial applications. In practice, this may be of most value for those bacterial species which are relatively slow-growing or difficult to identify by conventional methods.

The method described is simple, rapid, reliable, and informative. It is reliable because the identification result is based on analysis of independently cultured and analysed duplicates. It is simple and rapid because a microorganism culture can be easily prepared and analyzed within 20 minutes. The method is informative because it gives a quantitative estimate of the probability of an isolated microorganism belonging to a particular group.

The method would be applicable to hospital laboratories which require rapid identification of infectious agents to expedite appropriate and safe treatment. In industrial situations the method would improve process reliability and efficiency.

### EXAMPLE 2

### Using MRS to Distinguish Cryptococcomas from Gliomas in Rats and Cell Culture

1. Introduction

MRS was used to characterize clinical isolates of C. neoformans and a glioma cell-line in culture and in experimental rats. 1D and 2D ¹H MR spectra were acquired from fungi cultured in vitro (16 isolates of Cryptococcus neoformans, 3 of Candida albicans, 3 of Aspergillus fumigatus, 3 of Saccharomyces cerevisiae) and a C6 glioma cell line. Cerebral biopsies were obtained from healthy rats and animals with experimental infections or gliomas (19 healthy brains, 19 cryptococcomas and 20 gliomas). Unequivocal signal assignment was performed for cell suspensions as well as tissue samples using homo- and heteronuclear 2D correlation spectra (COSY, TOCSY, ¹H,¹³C-HSQC and HMBC). The results indicated that MR spectra from C. neoformans and cerebral cryptococcomas, but not from other fungi, healthy brain, or gliomas, were dominated by resonances from the cytosolic disaccharide α,α-trehalose. This spectral pattern was very different from that of gliomas, which was dominated by lipids and an increased choline/ creatine ratio, and from healthy brain. The results led to the conclusion that a remarkably high concentration of α,α-trehalose in relation to other MR-visible metabolites is diagnostic of C. neoformans. Cerebral cryptococcomas are a uncommon but serious manifestation of cryptococcosis in humans. Application of these results to the non-invasive diagnosis of cerebral cryptococcomas would reduce the risk and expense of unnecessary surgery or biopsy and expedite patient management.

2. Materials and Methods

a. In Vitro Cultures of Fungi and C6 Glioma Cell Line

Sixteen cryptococcal isolates were cultured in vitro and studied by MRS. These included 8 clinical isolates of C. neoformans serotype A (clinical isolates from lung, blood, CSF and brain), 7 isolates of serotype B (clinical (brain and CSF), veterinary and environmental isolates) and 1 clinical isolate of the teleomorph of C. neoformans, Filobasidiella neoformans var. bacilliformis (American Type Culture Collection 32609). Other fungi which were cultured in vitro and studied by MRS, included 3 each of the yeasts Candida albicans (clinical isolates) and Saccharomyces cerevisiae (environmental isolates and type cultures) and the fungus Aspergillus fumigatus (clinical isolates). Yeasts were identified biochemically using the API 20C AUX system (BioMerieux, March l'Etoile, France). Cryptococci were biotyped (45) and serotyped (Crypto Check agglutination test, Iatron Labs). Fungi were cultured for 24-48 h on Sabouraud's dextrose agar (SDA, Difco Labs, Detroit, Mich., USA), then either in Brain Hear Infusion broth at 30° C. (A. fumigatus) or in yeast nitrogen broth (Difco) containing 1% glucose, buffered at pH 7. 0 with 0.345% w/ v MOPS (Sigma Chemical Co., St. Louis, Mo., USA), at 27, 30 and/or 37° C., C6, a rat glioma cell line, was maintained as described (51) and used within 3-30 passages. Immediately before use, logarithmic phase fungal cells, or C6 glioma cells, were washed and resuspended in dulbecco's phosphate-buffered saline (PBS, Difco) for animal models or in PBS made up with 99.5% deuterium oxide (D₂O for MRS.

Culture conditions for the growth of one isolate of C. neoformans (Mc Bride strain) were varied to test the effect of stress on MR-visible metabolite profiles. The isolates were cultured in buffered yeast nitrogen broth (Difco Labs, Detroit, Mich., USA) containing 10 MM glucose. The following parameters were varied: incubation temperature (27, 35 and 42° C.), pH (5 and 7), glucose concentration (1, 10, 50 mM), substitution of glucose with mannose (10M) or sucrose (10 mM) and prolonged incubation in glucose-free medium (0-100 hr).

b. Animal Studies

Three isolates of C. neoformans serotype B (WM276, WM430, Mc Bride) and the C6 glioma cell line were used for animal experiments. Male Wistar-furth and female Fischer 344 rats (150-250 g, Animal Research Council, Perth, Wash.) were anaesthetized by inhalation of 4% halothane in 100% oxygen prior to intraperitoneal injectin of ketamine (11.6 mg/kg, Apex Laboratories, Sydney) and xylazine (1.2 mg/kg, Apex Laboratories, Sydney), and allowed to breathe room air spontaneously. For induction of brain lesions, the animal head was fixed in a sterotactic frame (David Kopf Instruments, Tajunga, Calif., USA); 5 μl of a suspension of C. neoformans serotype B, or c6 glioma cells, was injected through a straight, flat-ended 26 gauge needle at a rate of 3-6 μl/min. Preliminary experiments established that 5×10⁴ cfu of cryptococci suspended in a volume of 5 μl , and 1×10⁶ C6 cells, also in a volume of 5 μl, induced lesions of at least 3 mm in diameter, when harvested 6-12 days (cryptococcomas, n=18 rats) or 12-30 days (gliomas, n−26 rats) post-operatively (data not shown). Optimal coordinates for microinjection were; 2.0 mm below dura, 3.0 mm lateral, and 2.4 mm anterior-posterior relative to ear bar zero. Using these coordinates, rats were injected for the MR study with the C. neoformans serotype B isolate Mc Bride (cryptococcomas, n=20, gliomas, n=19; control, n=19). Control tissue was obtained from saline-injected rats. At appropriate times, rats were sacrificed, the brain was removed and cut transversely at the site of the lesion. Brain tissue was fixed in formalin, and embedded in paraffin. 7 μm sections were taken and stained with haematoxylin and cosin or periodic acid-Schiff reagent (PAS) for light microscopy. Brain tissue samples (diameter up to 4 mm) from each of the animals with cryptococcomas or gliomas and from controls were suspended in PBS/D₂O, snap-frozen in liquid nitrogen, and stored at 70° C. for up to 4 months for MRS analysis.

Animal experimentation was carried out according the Australian National Health and Medical Research Council Guidelines and with ethical approval from the University of Sydney Animal Ethics Committee.

7. MR Experiments

¹H MR spectra were obtained on a Bruker Avance 360 MHz spectrometer equipped with a 5-mm {¹H, ¹³C} inverse-detection dual-frequency probe. The temperature was maintained at 37° C. Residual water signal was suppressed by selective gated irradiation (46) or by selective excitation using pulse field gradients (47). Chemical shifts were referenced to external sodium 3-(trimethylsilyl) propanesulfonate (TSP) at 0.00 ppm or internal water (4.65 ppm), respectively. 1D ¹H MR spectra were acquired with a spectral width 3600 Hz, time domain 8k, 128 or 256 acquisitions, relaxation delay Is. A line broadening of 1 or 3 Hz was applied for cell culture and tissue samples, respectively, prior to Fourier transformation. Resonance ratios from fully relaxed ¹H MR spectra were used for comparison of cell types. Packed cell suspensions and samples were spun at 20 Hz to avoid the cells settling in the MR tube. A relaxation delay of 5 s was applied to allow full relaxation.

Two-dimensional MR spectra were acquired for unequivocal signal assignment. {¹H, ¹H} COSY experiments were performed in magnitude-mode (48). Acquisition parameters were: sweep width in t₂ 3600 Hz, t₂ time domain 2k, 256 increments of 32 or 48 acquisitions each, relaxation delay 1 s. Sine-bell window functions were applied in the t₁ dimension, and Gaussian-Lorentzian window functions were applied in the t₂ dimension. Zero filling was used to expand the data matrix to 1 K in the t₁ dimension. Cross-peak volumes were determined as described (49).

TOCSY spectra with mixing times of 40 ms and 120 ms were acquired with 256 increments of 2K data points and 48 scans per increment for confirmation of assignments (1). {¹H, ¹³C} one-bond shift correlation spectra were obtained in the ¹H detection mode using an HSQC pulse sequence (50) for some samples to confirm signal assignments. The ¹H MR spectra width was 3600 Hz and the ¹³C NMR spectral width was 15000 Hz. ¹³C MR decoupling during acquisition was achieved by GARP-1 (8). The evolution time (t₁) was incremented to obtain 256 FIDs, each of 80 acquisition and consisting of 2K data points. The relaxation delay was 1 s. A sine-bell function was applied in the t₂ dimension and a Gaussian-Lorentzian function was applied in the t₁ dimension. Zero filling to 1 K was used in the t₁ dimension prior to Fourier transformation.

¹H MR spectra and 2D {¹H, ¹H} COSY were acquired from all fungal isolates, the C6 glioma cell line and rat brain samples (20 with cryptococcomas, 19 with gliomas and 19 controls). Signal assignment was confirmed by TOCSY and HSQC for at least one isolate or sample of each of the four fungal species, the C6 glioma cell line and the different brain biopsy samples.

8. Results

a. Cell Culture Studies

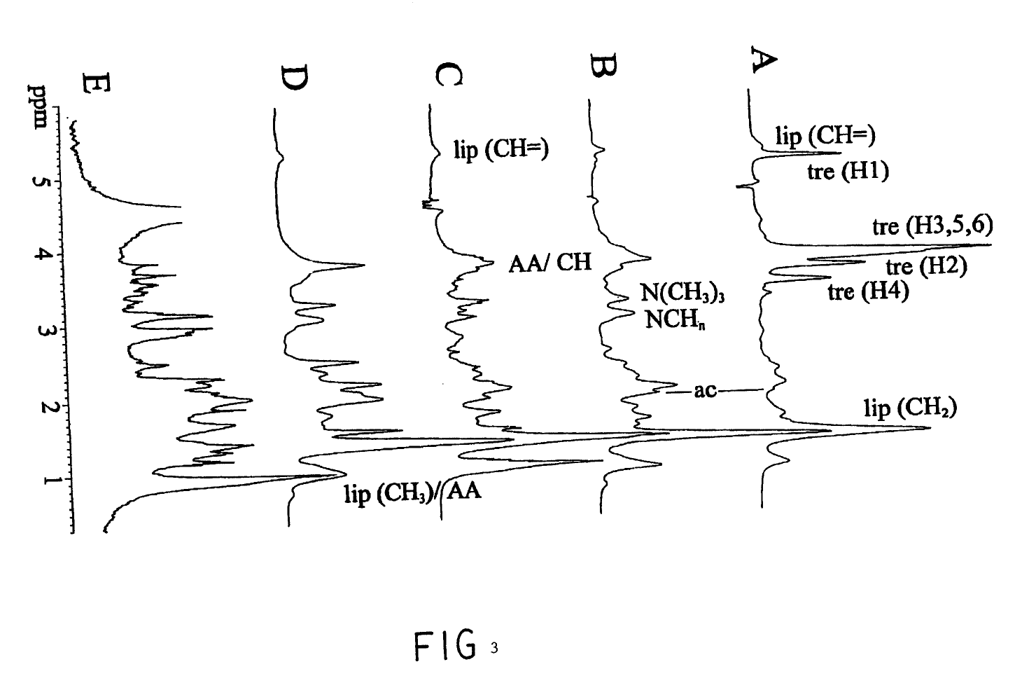

Typical one (1D) and two-dimensional (2D) MR spectra from fungi cultured in vitro and the C6 glioma cell line are compared in FIG. 3. Major cross-peaks from the 2D spectra are summarized in Table 5. Resonances were assigned either by comparison with published data (32, 40, 44, 51, 52) or by primary analysis of COSY, TOCSY and HSQC spectra. Resonances listed in Table 5 and shown in FIG. 3 were present in spectra from all isolates of the respective fungi and the C6 glioma cell line. Resonance intensities varied between isolates and shown in Table 6.

b. C. neoforman cultured in vitro

One-dimensional and 2D COSY MR spectra of C. neoformans were dominated by resonances from lipid and a,a,-trehalose signals. The spectral pattern of lipids was distinctive with chemical shifts at 0.90 ppm, 1.30 ppm, 1.60 ppm, 2.00 ppm, 2.30 ppm and 5.38 ppm (51). Resonances at 3.46 ppm, 3.66 ppm, 3.77-3.85 ppm and 5.19 ppm were assigned to a,a,-trehalose using HSQC spectroscopy on the cell suspensions. The respective assignments were: h1-C1 (5.19-93.50 ppm), H2-C2 (3.66-71.50 ppm), H3-C3 (3.86-73.0 ppm), H4-C4 (3.46-70.00 ppm), H5-C-5 (3.83-72.50 ppm) and H6-C6 (3.78 & 3.88/ 61.00 ppm). Less intense cross-peaks from amino acid residues [lysine (lys), alanine (ala), threonine (thr) and glutamate/glutamine (glu/gln)] and ethanol were evident in some strains (summarized in Tables 5 and 6). The dominant MR visible extracellular metabolities are acetate (1.92 ppm) and ethanol (1.16 and 3.63 ppm).

c. Effect of Stress on Cryptococcal Cells

Since trehalose has been reported to protect fungi against adverse conditions (heat, desiccation, osmotic and oxidative stress etc.) (54-57) consideration was given to the possibility that the MR-visible trehalose could vary with culture conditions. The effect of temperature, pH, glucose concentration, substitution of glucose with other sugars and incubation time as specified in Materials and Methods was tested. The respective resonance ratios varied by no more than a factor of four relative to standard conditions (data not shown). Lipid and trehalose signals remained dominant in ID and COSY spectra, irrespective of culture conditions. d. MRS of other fungi cultured in vitro MRS of two other clinically important, pathogenic fungi, Candida albicans and Aspergillus fumigatus, and the yeast Saccharomyces cerevisiae were investigated, and found to be different from C. neoformans. The 1D and 2D COSY spectra of C. albicans (FIG. 3b) and S. cerevisiae (FIG. 3d), revealed dominant lipids, whereas those from A. fumigatus (FIG. 3c) were characterized by resonances from amino acid residues and carbohydrates. Carbohydrates resonances from these fungi were of a much lower intensity than those from C. neoformans and could not be assigned to specfic monosaccharide residues. Very small amounts of MR-visible a,a-trehalose (approximately 20 times lower than in C. neoformans) were identifiable only in the COSY spectra of two of the three strains of S. cerevisiae and none in C. albicans, if exposed to high temperatures (37-43° C.). Ethanol was identified in the two yeast species. Acetate was visible in the spectra from the culture supernatants from C. albicans and A. fumigatus (data not shown).

e. C6 glioma Cell Line

The spectra of suspended C6 glioma cells cultured in vitro (FIG. 3e, summarized in Tables 3 and 4) were dominated by resonances from amino acids. The 2D spectra of C6 cells revealed no carbohydrate cross-peaks. Intense cross peaks arising from choline (chol), phosphocholine (PC) and glycerol-phosphocholine (GPC) and relatively high amounts of taurine (tau) and the amino acid residues leucine (leu) and glu/gln were present, as has been reported for tumour cell lines (51). The resonance ratio of 3.25 to 3.05 ppm, representing choline- and creatine-containing compounds, respectively, was much higher than that in the spectra from fingi (Table 6).

f. Animal Studies

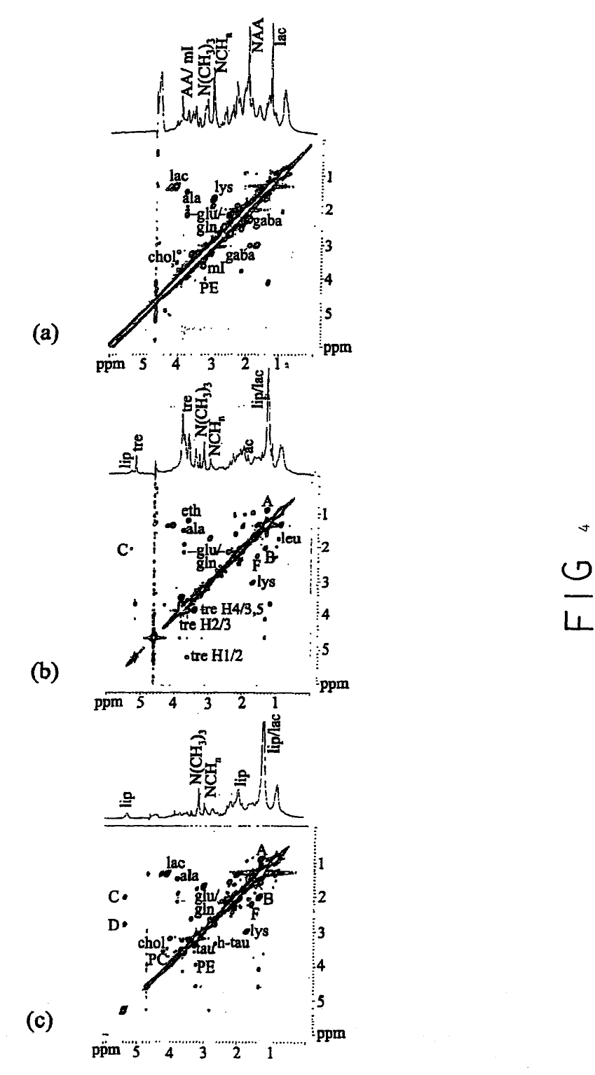

Histopathology of biopsy samples from rat brains showed that the biomass of cryptococcomas was comprised predominantly of cryptococci, as is seen in human infection and verified the pathology of tumours grown in the rat model (data not shown). Representative 1D ¹H MR and 2D COSY spectra from control rats, rats with cerebral cryptococcoma and rats with gliomas are shown in FIG. 4. Resonance ratios are summarized in Table 6.

Spectra from control brain tissue were dominated by N-acetyl aspartate (NAA) at 2.00 ppm. Characteristic signals of lower intensity included composite peaks at 2.0-2.2 ppm (glutamine/glutamate), at 3.0 ppm (creatine, phosphocreatine, (γ-amino) butanoic acid (GABA) and lys residues); at 3.2 ppm [N(CH₃)₃ groups of choline, PC and GPC etc] and at 3.6 to 3.9 ppm (Ha of amino acid residues and myo-inositol), amino acids GABA, chol, Pcand GPC signals were also present in COSY spectra. Lactate signals of variable intensity were found at 1.3 ppm, resulting from anaerobic metabolism occurring in the time between excision and freezing.

Cryptococcoma gave 1D and COSY MR spectra with the typical pattern of cryptococcal trehalose and lipids described above. The intensity of the lipid resonance at 5.38 ppm to the trehalose resonance at 5.19 ppm varied over a wide range (Table 6). Furthermore, the 3.2:3.0 ppm resonance ratio was elevated compared with normal brain tissue. The NAA signal decreased dramatically and was undetectable in some samples. Other resonances not observed in normal brain tissue arose from acetate (1D spectra) and ethanol (2D COSY) in some, but not all spectra. Also, a distinct cross peak arose from GPC, which was of much higher intensity than in control and glioma spectra. The myo-inositol and GABA cross-peak intensities in the COSY spectra were reduced relative to the amino acid cross-peaks when compared with those in control brain tissue.

Spectra from tumour biopsies were dominated by lipid signals and an increased resonance ratio at 3.20:3.00 ppm, which is consistent with many reports in the literature (32, 58, 59). The relative increase in lipid signal intensities and the elevation in the 3.2:3.0 ppm ratio was, for most samples, much larger than the increase found in cryptococcomas (Table 6). Resonance ratios for the lipid varied. NAA remained undetectable in many tumour specimens, indicative of absent neuronal activity. The only cross-peaks apart from lipids, that increased relative to other amino acid residue cross-peaks (e.g. lys, leu, etc.) were those of taurine (3.28-3.50 ppm), choline (3.50-4.07 ppm), PC (3.61-4.19 ppm) and phosphoethanolamine (3.22-3.98 ppm).

9. Discussion

C. neoformans was distinguished unequivocally from other yeasts, the filamentous fungus A. fumigatus and C6 glioma cells by MRS, due to an abundance of the non-reducing disaccharide, trehalose. These differences were recorded in spectra from cells cultured in vitro, as well as from affected tissue from rat cortex, where the diagnosis was confirmed histologically. MRS has thus provided a means of distinguishing cryptococcomas from healthy brain and brain tumour tissue in biopsy samples.

(C. neoformans in infected tissued is surrounded by a capsule which typically occupies many times the volume of the fungal cell. This capsule is composed predominantly of glucuroxylomannans (GXM) in a loosely-woven fibrillar configuration (42, 43). None of the cell-associated GXM from samples sultured in vitro, or from tissue biopsies, were MR-visible, indicating that native capsular polysaccharides are not sufficiently mobile to be visible using MRS.

In contrast, the cytosolic compound, trehalose, was identified and present in very large amounts. Trehalose is present in yeasts, and other fungi (60) and is therefore not a unique characteristic of c. neoformans, per se. It is an important protectant induced by conditions of heat (57), osmotic stress (61), dehydration (56), dessication and other (for review see (54) and (55)). Trehalose levels in C. neoformans cultured at 25° C., however, exceeded those n S. cerevisiae under conditions of heat stress (37° C.) by at least 20 times. Altered culture conditions did not reduce the intensity of the trehalose signal in C. neoformans.

The large amount of trehalose relative to other MR-visible compounds in the spectra of C. neoformans defines trehalose as one marker that can be used to distinguish C. neoformans from other fungi. It is possible that such high levels of trehalose in cryptococci are an evolutionary response to environmental stress, particularly temperature, dehydration and starvation. Adaptation to survival and growth at physiological temperatures, a recognized virulence determinant of C. neoformans (62) is consistent with high intracellular concentrations of trehalose.

Bacterial metabolities, but not α,α-trehalose have been identified by ¹H MRS in pus samples from patients with bacterial brain abscesses (5, 34, 35, 39). Ethanol, a product of glucose fermentation in yeasts, was reported to be present in the CSF of a patient with cryptococcal meningitis (63). The predominant extracellular metabolites (acetate, ethanol) found in the present study and by Bubb et al. (44) are not suitable for definitive in vivo or ex vivo diagnosis of cryptococcomas as they are also produced by other pathogenic microorganisms (5). The distinctive acetate signal present in many crytococcomas, but not healthy or neoplastic tissue, may, however, be a useful diagnostic indicator of infection. Acetate is produced by bacteria and has been identified by MRS in bacterial abscesses (34, 35, 39) as well as in this study, in C. neoformans and cryptococcomas.

The results discussed above indicate that MRS can distinguish unequivocally between healthy brain, and experimental crytoccocomas and gliomas from rats. The high level of MR visible α,α-trehalose recorded from cyptococcomas provides a basis for the path for the pathological diagnosis of cerebral cyptococcomas. When this method is applied to in vivo diagnosis of cerebral cryptococcomas in humans, cerebral cyptococcomas will no longer be mistaken for malignancies by conventional imaging modalities. An early and correct diagnosis will reduce the high morbidity and mortality (27) that occurs when diagnosis is delayed. Using in vivo MRS as a non-invasive method of diagnosis of infective lesions in the brain will reduce the risk and expense of unnecessary surgery or biopsy and expedite patient management decision.

### EXAMPLE 3

### Identification of Pathogenic Fungi

1. Microorganisms

Two hundred and five cultures of the pathogenic yeasts Candida albicans, C. parapsilosis, C. tropicalis, C. krusei and C. glabrata were cultured for 48 h on Sabouraud's dextrose agar at 30° C. 69 and 70 isolates of the pathogenic yeast Cryptococcus neoformans var. neoformans and var. gattii, respectively, were cultured for 48 h on Sabouraud's dextrose agar at 37° C. Yeasts were identified biochemically using the API 20C AUX system (BioMerieux, Marcy l'Etoile, France). Cryptococci were biotyped and serotyped (Crypto Check agglutination test, latron Labs). Additionally, PCR fingerprinting was used to compare the genotype of the respective species. Colonies were scraped from the plates and resuspended in PBS/ D20 immediately before the MR experiments.

2. MR spectroscopy

MR spectra were acquired on a 360 MHz Bruker Avance NMR spectrometer using an 5 mm {1H, 13C} inverse detection probe. Signal assignment was performed using COSY, TOCSY (tₘ=40, 150 ms), 1H, 13C HSQC (optimized to 1J=130 Hz) and 1H, 13C HMBC (optimized to n J=7 Hz).

3. Statistical Classification Strategy (SCS)(9):

The five Candida species were subdivided into two groups containing cultures from 2 or 3 species, respectively. Pair-wise classification was performed to distinguish between these groups and later to distinguish between the species in each group. Pair-wise classification was performed for the two C. neoformans varieties. Magnitude MR spectra were normalized to the total integral and analyzed by a genetic-algorithm-based Optimal Region Selector(⁶⁶) to identify three maximally discriminatory subregions, using rank-ordered first derivatives of the spectra(⁹). Using these three regions, a Linear Discriminant Analysis based classifier was developed. The robustness of the classifier was tested by bootstrap-based(67) crossvalidation (1000 repeats)(⁹). Class assignment was called crisp if class probabilities were >0.75.

4. Results

MR spectra of all yeasts (Candida species as well as Cryptococcus varieties) were dominated by signals of lipids, carbohydrates (trehalose, glucose), polyols (glycerol, mannitol, glucitol and others), ethanol and amino acid residues.

a. Candida Species:

Visual analysis of the ID MR spectra of Candida species allowed a distinction into two groups: (A) C. krusei and C. glabrata; (B) C. albicans, C. parapsilosis and C. tropicalis. These differences were mainly due to a higher carbohydrate and ethanol content in group A. These groups are consistent with a phylogenetic tree based on partial actin gene sequences 64. Pair-wise classification between groups A and B and between species in each group resulted in overall accuracies of up to 99%.

b. Cryptococcus Varieties:

Both ID and 2D MR spectra from the C. neoformans varieties were indistinguishable by visual inspection. However SCS on 1D MR spectra distinguished the varieties neoformans and gattii with an accuracy of 98.6% using a pair-wise statistical classification strategy.

5. Discussion

MRS data analyzed by SCS were compared with currently used biochemical identification tests and molecular biological methods (PCR fingerprinting). Different species and varieties of fungi could be identified by applying a Statistical Classification Strategy to MR spectra. Thus identification of fungi was possible below the taxonomic rank of species with a high degree of accuracy in these particular systems. The rapidity with which the SCS algorithm could analyze the MR data, collected and processed in less than 10 minutes, makes this an attractive option for routine testing in microbiology laboratories. Thus, according to the invention, SCS-based analysis of MR data can identify both species and varieties of fungi faster than other conventional methods, with a high degree of accuracy.

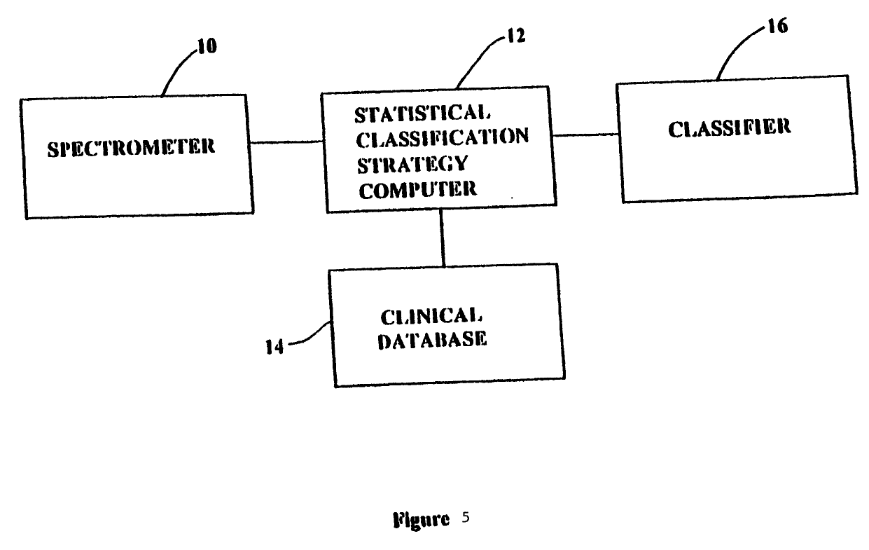

FIG. 5 shows a spectrometer 10, which may be a Bruker Avance 360 MHz MR spectrometer, with equipped computer. The statistical classification strategy (SCS) computer 12 stores the SCS and other programs described therein. The clinical data base includes the information from the data acquisition and the identity of known microorganisms and the like which is used by the computer 12 to develop the classifier 16.

Although the examples described relate to in vitro analysis, the present invention can be used for in vivo analysis, in which case a more powerful magnet may be obtained in the spectrometer.

Although at least one embodiment of the invention has been shown and described, variations and modifications may occur to those skilled in the art. The invention is not limited to the preferred embodiment, and its scope is determined only by the appended claims.

### REFERENCES

1. Bax, A., and D. Davis. 1986. MLEV-17-based two dimensional homonuclear magnetization transfer spectroscopy. J. Magn.Reson. 65:355-360.

2. Bradley, E., and R. Tibshirani. 1993. An introduction to the bootstrap. Chapman & Hall, London.

3. Braun, S., H.-O. Kalinowski, and S. Berger 1998. 150 and More Basic NMR Experiments. Wiley-VCH, New York.

4. Cohen, J. 1968. Weighted Kappa: Nominal scale agreement with provision for scaled disagreement or partial credit. Psychol Bull. 70:213-218.

5. Delpassand, E. S., M. V. Chari, C. E. Stager, J. D. Morrisett, J. J. Ford, and M. Romazi. 1995. Rapid identification of common human pathogens by high-resolution proton magnetic resonance spectroscopy. J. Clin. Microbiol. 33(5): 1258-62.

6. Gadian, D. G. 1995. NMR and its Applications to Living Systems. Oxford Unversity Press, Oxford.

7. Goodacre, R., J. K. Heald, and D. B. Kell. 1999. Characterisation of intact microorganisms using electrosray ionisation mass spectrometry. FEMS Microbiol. Lett.

176(1):17-24.

8. Goodacre, R., E. M. Timmins, P. J. Rooney, J. J. Rowland, and D. B. Kell. 1996. Rapid Identification of Streptococcus and Enterococcus Species Using Diffuse Reflectance-Absorbance Fourier Transform Infrared Spectroscopy and Artificial Neural Networks. FEMS Microbiol. Lett. 140(2-3):233-239.

9. Hahn, P., 1. C. Smith, L. Leboldus, C. Littman, R. L. Somorjai, and T. Bezabeth. 1997. The classification of benign and malignant human prostate tissue by multivariate analysis of ¹H magnetic resonance spectra. Cancer Res. 57(16):3398-401.

10. Hardie, J. M., and R. A. Whiley. 1997. Classification and Overview of the Genera Streptococcus and Enterococcus. J. Appl. Microbiol. 83(Suppl S):S 1-S 11.

11. Kummerle, M., S. Scherer, and H. Seiler. 1998. Rapid and Reliable Identification of Food Borne Yeasts By Fourier-Transform Infrared Spectroscopy. Appl. Environ. Microbiol. 64(6):2207-2214.

12. Magee, J. 1993. Whole-organism Fingerprinting, p. 383-427. In M. Goodfellow and A. G. O'Donnell (ed.), Handbook of New Bacterial Systematics. Harcourt Brace, New York.

13. Mounford, C., R. Somorjai, L. Gluch, P. Malycha, C. Lean, P. Russell, M. Bilous, B. Barraclough, David Gillett, U. Himmelreich, B. Dolenko, A. Nikulin, and I. Smith. 2001. MRS on breast fine needle aspirate biopsy determines pathology, vascularization and nodal involvement. Br. J. Surg. (In press).

14. Naumann, D., V. Fijala, H. Labischinski, and G. Giebrecht. 1998. The rapid differetiation and identification of pathogenic bacteria using Fourier transform infrared spectroscopy.

Journal of Molecular Structure. 174:165-170.

15. Nelson, W., R. Manoharan, and J. Sperry. 1992. UV Resonance Raman Studies of Bacteria. Applied Spectroscopy Review. 27:67-124.

16. Pot, B., P. Vandamme, and K. Kersters. 1994. Analysis of electrophoretic whole-organism protein fingerprints., p. 493-521. In M. Goodfellow and A. O'Donnell (ed.), Chemical methods in prokaryotic systematics. John Wiley & Sons, Chichester.

17. Russell, P., C. Lean, L. Delbridge, G. May, S. Dowd, and C. Mountford, 1994.. Proton magnetic resonance and human thyroid neoplasia. I: Discrimination between benign and malignant neoplasms. Am. J. Med. 96:383-388.

18. Shaka, A., P. Barker, and R. Freeman. 1985. Computer-optimized decoupling scheme for wideband applications and low level operation. J. Magn. Reson. 64:547-552.

19. Somorjai, R., B. Dolenko, A. Nikulin, P. Nickerson, D. Rush, A. Shaw, M. Glogowski, J. Rendell, and R. Deslauriers. 2000. Distinguishing Normal from Rejecting Renal Allografts: Application of a Three-Stage Classification Strategy to MR and IR of Urine. Vibrational Spectroscopy. (in press).

20. Somorjai, R. L., B. Dolenko, A. K. Nikulin, N. Pizzi, G. Scarth, P. Zhilkin, W. Halliday, D. Fewer, N. Hill, I. Ross, M. West, I. C. P. Smith, S. M. Donnelly, A. C. Kuesel, and K. M. Briere. 1996. Classification of ¹H MR spectra of human brain neoplasms: the influence of preprocessing and computerized consensus diagnosis on classification accuracy. J. Magn. Reson. Imaging. 6(3):437-44.

21. Somorjai, R. L., A. E. Nikulin, N. Pizzi, D. Jackson, G. Scarth, B. Dolenko, H. Gordon, P. Russell, C. L. Lean, L. Delbridge, 1995. Computerized consensus diagnosis: a classification strategy for the robust analysis of MR spectra. I. Application to ¹H spectra of thyroid neoplasms. Magn. Reson. Med. 33(2):257-63.

22. Stager, C. E., and J. R. Davis. 1992. Automated systems for identification of microorganisms. Clin. Microbiol. Rev. 5(3):302-27.

23. Wallace, J. C., G. P. Raaphorst, R. L. Somorjai, C. E. Ng, M. Fung Kee Fung, M. Senterman, and I. C. P. Smith. 1997. Classification of ¹H MR spectra of biopsies from untreated and recurrent ovarian cancer using linear discriminant analysis. Magn Reson. Med. 38(4):569-76.

24. Willker, W., D. Leibfritz, R. Kerssebaum, and W. Bermel. 1993. Gradient selection in inverse heteronuclear correlation spectroscopy. Magn. Reson. Chem. 31:287-292.

25. Mitchell T G, Perfect J R. Crytococcosis in the era of AIDS-100 years after the discovery of Cryptococcus neoformans. Clin Micro Rev 1995; 8:515-548.

26. Speed B, Dunt D. Clinical and host differences between infections with the two varieties of Cryptococcus neoformans. Clin Infect Dis 1995; 21:28-34.

27. Mitchell D H, Sorrell T C, Allworth A M, Health C H, McGregor A R, Papanaoum K, Richards M J, Gottlieb T. Crytoccocal disease of the CNS in immunocompetent hosts:

influence of crytococccal variety on clinical manifestations and outcome. Clin. Infect. Dis 1995; 20:611-616.

28. Chen S C A., Sorrell T C, Nimmo G, Speed B, Currie B, Ellis D, Marriott D, Pfeiffer T, Parr D, Byth K. Epidemiology, and host and variety-dependent characteristics of infection due to Cryptococcus neoformans, in Australia and New Zealand. Clin. Infect. Dis. 2000; 31:499-508.

29. Fujita N K, Reynard M, Sapico F L, Guze L B, Edwards J E Jr. Crytococcal intracerebral mass lesions: the role of computed tomography and nonsurgical management. Ann Intern Med 1981; 94:382-388.

30. Andreula C F, Burdi N, Carella A. CNS crytococcosis in AIDS:spectrum of MR findings J. Comp Assisted Tomog 1993; 17,438-441.

31. Negendank W. Studies of human tumors by MRS: a review. NMR Biomed 1992; 5:303-324.

32. Remy C, Grand S, Lai ES, Belle V, Hoffmann D, Berger F, Ziegler A, Le Bas J F, Benabid A L, et al. 1H MRS of human brain abscesses in vivo an in vitro. Magn Reson Med 1995; 34:508-514.

33. Hagberg G. From magnetic resonance spectroscopy to classification of tumor-a review of pattern recognition methods. NMR Biomed 1998; 11:148-156.

34. Dev R, Gupta R K, Poptani H, Roy R, Sharma S, Hasain M. Role of in vivo proton magnetic resonance spectroscopy in the diagnosis and management of brain abscesses. Neurosurgery 1998; 2:37-42.

35. Kim S H, Chang K H, Song I C, Han M H, Kim, H C, Kang, H S, Han M C. Brain abscess and brain tumor-discrimination with in vivo H-1 MR spectroscopy. Radiology 1997; 204:239-245.

36. Grand Spassaro G, Ziegler A, Esteve F, Boujet C, Hoffman D, Rubin C, Segebarty C, Decorps M, LeBas J-F, Remy C. Necrotic tumor versus brain abscess: importance to amino acids detected at ¹H MR spectroscopy-initial results. Radiology 1999; 213:785-793.

37. Danielsen E R, Ross B D. Magnetic Resonance Spectroscopy Diagnosis of Neurological Diseases. New York: Marcel Dekker Inc., 1998.

38. Saunders D E, Howe F A, van der Boogaart A, McLean M A, Griffiths J R, Brown M M. Continuing ischemic damage after acute middle cerebral artery infarction in humans demonstrated by short-echo proton spectroscopy. Stroke 1995; 26, 1007-1013.

39. Remy C, Arus C, Ziegler A, Lai ES, Moreno A, Le Fur Y, Decorps M. In vivo, ex vivo, and in vitro one-and two dimensional nuclear magnetic resonance spectroscopy of an intracerebral glioma in rat brain: assignment of resonances. J. Neurochem. 1994; 62:166-179.

40. Delikatny E J, Russell P, Hunter J C, Hancock R, Atkinson K H, Van Haaften-Day C, Mountford C E. Proton M R and human cervical neoplasia: ex vivo spectroscopy allows distinction of invasive carcinoma of the cervix from carcinoma in situ and other preinvasive lesions. Radiology 1993; 188:791-796.

41. Makinnon W B, Barry P A, Malycha P L, Gilleft D J, Russell P, Lean C L, Doran S T, Barraclough B H, Bilous M, Mountford C E. Fine-needle biopsy specimens of benign breast lesions distinguished from invasive cancer ex vivo with proton M R spectroscopy. Radiology 1997; 204:661-666.

42. Casadevall A, Perfect J E. In Cryptococcus neoformans. Washington D. C., American Society for Microbiology Press, 1998.

43. Cherniak R, Sundstrom JB. Polysaccharide antigens of the capsule of Crytococcus neoformans. Infect. Immun 1994; 62:1507-1512.

44. Bubb W A, Wright L C, Cagney M, Santangelo R T, Sorrell T C, Kuchel P W. Heteronuclear NMR studies of metabolites produced by Cryptococcus neoformans in culture media: Identification of possible virulence factors. Magn Reson Med 1999; 42:442-453.

45. Kwon-Chung K J, Polacheck I, Bennett J E. Improved diagnostic medium for separation of Cryptococcus neoformans var. neoformans (serotypes A and D) and Cryptococcus neoformans var. gatti (serotypes B and C). J Clin Microbiol 1982; 15-535-537.

46. Jesson J P, Meakin P, Kneissel G, Homonuclear decoupling and peak elimination in Fourier transform nuclear magnetic resonance. J Am Chem Soc 1973; 95:618-620.

47. Tsang-Lin Hwang, Shaka A J. Water suppression that works. Excitation sculpting using arbitrary waveforms and pulse field gradients. J Magn Reson 1995; 112:275-279.

48. Aue W P, Bartholdi E, Ernst R R. Two-dimensional spectroscopy. Application to nuclear magnetic resonance. J Chem Phys 1976; 64:2229-2246.

49. Lean C L, Mackinnon W B, Mountford C E. Fucose in 1H COSY spectra of plasma membrane fragments shed from human malignant colorectal cells. Magn Reson Med 1991; 20:306-311.

50. Palmer III A G, Cavanagh J, Wright P E, Rance M. Sensitivity improvement in proton-detected two-dimensional heteronuclear correlation NMR spectroscopy. J Magn Reson 1991; 93:151-170.

51. Lean C L, Mackinnon W B, Delikatny E J, Whitehead R H, Mountford C E. Cell-surface fucosylation and magnetic resonance spectroscopy characterization of human malignant colorectal cells. Biochemistry 1992; 31:11095-11105.

52. Kalinowski H O, Berger S, Braun S. ¹³C-NMR-Spektroskopie. Stuttgart: Georg Thieme Verlag 1984.

53. Barton J K, Den Hollander J A, Hopfield J J, Shulman R G. 13C nuclear magnetic resonance study if trehalose mobilization in yeast spores. J. Bacteriol 1982; 151:177-185.

54. Wiemken A. Trehalose in yeast, stress protectant rather than reserve carbohydrate. Antonie Leeuwenhoek 1990; 58:209-217.

55. Van Laere A J. Trehalose, reserve and/or stress metabolite. FEMS Micorbiol Rev 1989; 63:201-210.

56. Crowe J H, Hoekstra F A, Crowe L. M. Anhydrobiosis. Annu Rev Physiol 1992; 54:579-599.

57. De Virgillo C, Hottiger T, Domingnez J, Boller T, Wiemken A. The role of trehalose synthesis for the acquisition of thermotolerance in yeast. I. Genetic evidence that trehalose is a thernoprotectant. Eur J Biochem 1994; 219:179-186.

58. Descord M, Remy C, Bourgeois D, Jacrot M, van Kienlin M, Benabid A L. Localised spectroscopy of rat brain tumors. Magn Reson Med 1989; 2:129-151.

59. Ross B D, Merkle H, Hendrich K, Staewen R S, Garwood M. Spatially localized in vivo 1H magnetic resonance spectroscopy of an intracerebral rat glioma. Magn Reson Med 1992; 23:96-108.

60. Thevelein J M. Regulation of trehalose metabolism and its relevance to cell growth and function. 395-420. In Brambl R, Marzluf, G A, eds. The Mycota. Vol 3. Berlin: Springer Verlag 1996.

61. Hengge-Aronis R, Klein W, Lange R, Rimmele M, Boos W. Trehalose synthesis genes are controlled by the putative sigma factor encoded by rpoS and are involved in stationary-phase thermotolerance in Escherichia coli. J. Bacteriol 1991; 173:7918-7924.

62. Kwon-Chung K J, Rhodes J C. Encapsulation and melanin formation as indicators of virulence in Crytococcus neoformans. Infect Immun 1986; 51:218-223.

63. Dawson D M, Taghavy A. A test for spinal-fluid alcohol in torula meningitis. New Eng J Med 1963; 269: 1424-1425.

TABLE 1
Classification and Identification Results With Optimised Classifier

Repeat cultures
1 2 3 4 5 6

| Species Group | Lab. No. | Classification probability (a) | Error group (b) | ID Result (c) |
|---|---|---|---|---|
| E. faecalis | ATCC 29212 | 100, 100 100, 100 100, 100 100, 100 100, 100 100, 99 | | c |
| 18 isolates | 083-1246 | 96, 98 | | c |
| 60 cultures | 175-1753 | 100, 100 | | c |
| | 184-0712 | 100, 100 100, 100 | | c |
| | 184-0721 | 100, 100 62, 97 100, 100 | | c |
| | 200-1831 | 100, 98 77, 87 | | c |
| | 200-2616 | 100, 100 99, 100 | | c |
| | 206-0685 | 100, 100 98, 94 | | c |
| | 270-2132 | 100, 100 | | c |
| | 273-2358 | 100, 100 | | c |
| | 282-0250 | 100, 100 | | c |
| | 282-0407 | 100, 100 | | c |
| (gallinarum) | 182-2747 | 98, 96 98, 100 | | c |
| (gallinarum) | 4/14/1956 | 100, 100 | | c |
| (gallinarum) | 4/14/1953 | 100, 100 | | c |
| (casseliflavus) | 4/14/1958 | 87, 76 | | c |
| (casseliflavus) | 4/14/1952 | 100, 100 | | c |
| (casseliflavus) | 207-2246 | 100, 100 | | c |
| S. aureus | ATCC 25923 | 100, 100 100, 100 100, 100 100, 100 100, 100 100, 100 | | c |
| 18 isolates | 008-1690 | 100, 100 | | c |
| 56 cultures | 040-2754 | 100, 100 | | c |
| | 099-1094 | 89, 91 | | c |
| | 124-2873 | 100, 100 94, 100 | | c |
| | 127-2131 | 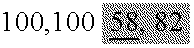 | S. epidermidis | INDETERMINATE |
| | 127-2297 | 100, 100 100, 100 | | c |
| | 242-2881 | 100, 100 | | c |
| | 261-1095 | 100, 100 | | c |
| | 271-0835 | 99, 100 | | c |
| | 281-2429 | 100, 100 | | c |
| | 29213 | 100, 100 | | c |
| | 319-2410 | 100, 100 | | c |
| | 320-2161 | 100, 100 | | c |
| | 320-2356 | 72, 100 100, 100 100, 100 | | c |
| | 323-0934 | 100, 100 | | c |
| | 323-1573 | 100, 100 | | c |
| | 338-1348 | 100, 100 | | c |
| S. epidermidis | ATCC 12228 | 100, 100 100, 100 100, 98 100, 100 50, 84 | | INDETERMINATE |
| 14 isolates | 003-1283 | 100, 100 75, 100 | | c |
| 40 cultures | 141-1667 | 100, 100 | | c |
| | 162-2710 | 100, 100 | | c |
| | 170-1085 | 100, 99 | | c |
| | 174-2177 | 100, 89 | | c |
| | 177-1320 | 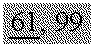 | S. aureus | INDETERMINATE |
| | 270-0170 | 100, 100 | | c |
| | 281-0122 | 100, 100 | | c |
| | 289-1072 | 95, 99 | | c |
| | 319-1923 | 100, 100 | | c |
| | 323-1622 | 100, 100 | | c |
| | 326-2592 | 100, 100 | | c |
| | 327-2569 | 100, 100 | | c |
| S. agalactiae | 048-1676 | 100, 100 | | c |
| 15 isolates | 159-2821 | 100, 100 | | c |
| 36 cultures | 165-1046 | 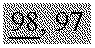 | S. milleri group | INDETERMINATE |
| | 176-0797 | 100, 100 100, 100 100, 100 | | c |
| | 183-2646 | 100, 100 | | c |
| | 208-2835 | 100, 100 | | c |
| | 242-1786 | 100, 100 | | c |
| | 260-1829 | 100, 100 | | c |
| | 269-0712 | 100, 100 | | c |
| | 269-1137 | 100, 100 | | c |
| | 269-1160 | 100, 100 | | c |
| | 270-1106 | 100, 100 | | c |
| | 285-2806 | 100, 100 | | c |
| | 290-1094 | 100, 100 | | c |
| | 291-1523 | 100, 100 | | c |
| S. milleri group | 073-0596 | 100, 100 | | c |
| 11 isolates | 097-1166 | 100, 100 | | c |
| 30 cultures | 141-0714 | 100, 100 99, 100 | | c |
| | 141-1834 | 99, 100 | | c |
| | 150-1172 | 100, 100 | | c |
| | 164-0507 | 98, 89 | | c |
| | 185-1175 | 100, 100 100, 99 | | c |
| | 291-0591 | 98, 96 | | c |
| | 291-1767 | 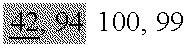 | S. pneumoniae | INDETERMINATE |
| | 349-2488 | 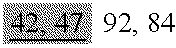 | S. pneumoniae, S. pyogenes | INDETERMINATE |
| | 408-0803 | 100, 100 | | c |
| S. pneumoniae | ATCC 6305 | 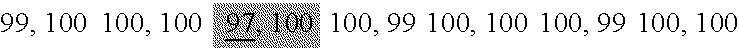 | S. pyogenes | INDETERMINATE |
| 15 isolates | 221-2745 | 78, 74 | | c |
| 42 cultures | 221-2755 | 100, 100 | | c |
| | 230-2817 | 100, 99 | | c |
| | 234-1207 | 91, 100 | | c |
| | 235-2193 | 94, 100 | | c |
| | 241-1187 | 100, 92 | | c |
| | 259-1456 | 100, 100 | | c |
| | 272-0604 | 100, 98 | | c |
| | 278-1723 | 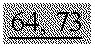 | S. pyogenes (both) | INDETERMINATE |
| | 278-1727 | 97, 100 | | c |
| | 324-1010 | 100, 100 | | c |
| | 404-0191 | 100, 98 | | c |
| | 467143 | 100, 100 | | c |
| | 480837 | 72, 90 | | c |
| S. pyogenes | ATCC 19615 | 98, 99 99, 100 100, 100 100, 100 100, 100 100, 100 | | c |
| 13 isolates | 162-1915 | 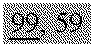 | S. pneumoniae | INDETERMINATE |
| 48 cultures | 213-0136 | 100, 70 100, 100 | | c |
| | 221-1798 | 99, 100 99, 99 | | c |
| | 221-2985 | 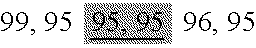 | E. faecalis (both) | MISIDENTIFICATION |
| | 223-2690 | 99, 100 100, 100 | | c |
| | 235-3096 | 94, 87 | | c |
| | 236-1570 | 98, 100 | | c |
| | 260-2388 | 88, 100 93, 96 | | c |
| | 312-2457 | 99, 99 | | c |
| | 3/12/2006 | 98, 99 | | c |
| | 326-0413 | 95, 100 | | c |
| | 3-61-70 | 89, 90 | | c |

a. Numbers show classification probabilities (%) for each spectrum of duplicate cultures.
Classification probabilities less than 75% are shown in bold typeface.
Shaded areas show where one or both of the spectra of duplicate cultures is misclassified.
Misclassifications are underlined.
b. The error group is the species to which a spectrum was incorrectly assigned.
c. Isolate Identification Result (c = correct)

TABLE 2
Summary of Classification and Identification Results

| Classification Type | Count | % of total |
|---|---|---|
| Correct | 294 | 94.2 |
| Indeterminate | 13 | 4.2 |
| Misclassification | 5 | 1.6 |
| Total: | 312 | 100.0 |
| Identification Type | Count | % of total |
| Correct | 146 | 93.6 |
| Indeterminate | 9 | 5.8 |
| Misidentification | 1 | 0.6 |
| Total: | 156 | 100.0 |

TABLE 3
Integral regions and most significant contributing metabolites

| | Range | |
|---|---|---|
| Region | (ppm) | Metabolites with resonances in this region |
| 1 | 4.00-3.81 | AA, betaine, GPC, GPE, EA |
| 2 | 3.81-3.70 | AA, glycerol, G3P |
| 3 | 3.70-3.50 | AA, GPC, glycine, choline, inositol |
| 4 | 3.50-3.34 | taurine, GPE, tryptophan |
| 5 | 3.34-3.10 | histidine, tyrosine, taurine, phenylalanine, betaine, GPC, choline, inositol, PA, EA |
| 6 | 3.10-2.88 | lysine, histidine, tyrosine, asparagine, PA |
| 7 | 2.88-2.61 | aspartate, asparagine, methionine |
| 8 | 2.61-2.42 | succinate |
| 9 | 2.42-2.22 | valine, glutamine, glutamate, valine, succinate |
| 10 | 2.22-1.95 | isoleucine, glutamine, glutamate, methionine, PA, N-acetyl compounds |
| 11 | 1.95-1.80 | acetate, lysine, isoleucine |
| 12 | 1.80-1.58 | leucine, lysine |
| 13 | 1.58-1.40 | lysine, alanine |
| 14 | 1.40-1.23 | lactate, isoleucine, threonine |
| 15 | 1.23-1.08 | (no metabolites identified) |
| 16 | 1.08-0.75 | valine, leucine, isoleucine |

Abbreviations:
AA, amino acid (non-specific);
PA, polyamine.
GPC, glycerol phosphocholine;
GPE, glycerol phosphoethanolamine;
EA, ethanolamine.

TABLE 4
Classification of Spectra of Enterococcus spp.

| | | Repeat cultures | |
|---|---|---|---|
| | | 1 2 3 4 5 6 | |
| Species Group | Lab. No. | Classification probability (a) | Error group (b) |
| E. faecalis | ATCC 29212 | 100, 100 100, 100 100, 100 100, 100 100, 100 100, 100 | |
| 12 isolates | 083-1246 | 100, 100 | |
| 44 cultures | 175-1753 | 100, 100 | |
| | 184-0712 | 100, 100 | |
| | 184-0721 | 100, 100 100, 100 100, 100 | |
| | 200-1831 | 100, 100 100, 100 | |
| | 200-2616 | 100, 100 100, 100 | |
| | 206-0685 | 100, 100 100, 100 | |
| | 270-2132 | 100, 100 | |
| | 273-2358 | 100, 100 | |
| | 282-0250 | 100, 100 | |
| | 282-0407 | 100, 100 | |
| E. gallinarum | 182-2747 | 100, 100 100, 100 | |
| 3 isolates | 4-14-56 | 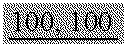 | E. faecalis (both) |
| 8 cultures | 4-14-53 | 100, 100 | |
| E. casseliflavus | 4-14-58 | 100, 100 | |
| 3 isolates | 4-14-52 | 100, 100 | |
| 6 cultures | 207-2246 | 100, 100 | |

Notes as for Table 1.

TABLE 5
Major components in the COSY spectra identified by their cross peak volumes.

| | | | | | | | | | tre | tre |
|---|---|---|---|---|---|---|---|---|---|---|
| | No. | A* | B* | C* | F* | chol | PC | GPC | (H1/H2) | (H2/H3) |
| Chemical shift | samples/ | 0.9- | 1.3- | 2.02- | 1.6- | 3.5- | 3.61- | 3.67- | 5.19- | 3.66- |
| (ppm) | isolates | 1.3 | 2.08 | 5.3 | 2.3 | 4.07 | 4.2 | 4.33 | 3.66 | 3.86 |
| CULTURES | | | | | | | | | | |
| C. neoformans | 16/16 | 3-4 | 3-4 | 2 | 3-4 | — | — | — | 3 | 4 |
| C. albicans | 5/3 | 4 | 4 | 3 | 4 | — | — | — | — | — |
| A. fumigatus | 3/3 | 2-3 | 2-3 | 1-2 | 2-3 | — | — | — | — | — |
| S. cerevisiae | 6/3 | 3 | 3 | 2 | 2-3 | — | — | — | — | 1^{Ψ} |
| C6 glioma cells | 3/1 | 1-3 | 1-3 | 1-2 | 2-3 | 1-2 | 2 | 1^{Ψ} | — | — |
| ANIMAL STUDIES | | | | | | | | | | |
| Control brain | 19 | 1 | — | — | — | 2 | 1-2 | — | — | — |
| Glioma | 19 | 4 | 4 | 2-3 | 3-4 | 3-4 | 1 | 2-3 | — | — |
| Cryptococcoma | 20 | 3 | 3 | 1 | 2 | 1 | — | 1-2 | 2 | 3-4 |

| | | tre | | | | | | | | |
|---|---|---|---|---|---|---|---|---|---|---|
| | No. | (H4/H3,5) | ala | lys | tau | inos | glu/gln | gaba | NAA | eth |
| Chemical shift | samples/ | 3.47- | 1.49- | 1.72- | 3.28- | 3.25- | 2.1- | 1.9- | 2.58- | 1.16- |
| (ppm) | isolates | 3.8 | 3.79 | 3.0 | 3.5 | 3.64 | 2.47 | 2.31 | 4.4 | 3.63 |
| CULTURES | | | | | | | | | | |
| C. neoformans | 16/16 | 4 | 1-2 | 1-3 | — | — | 1^{Ψ} | 1^{Ψ} | — | 1-2^{Ψ} |
| C. albicans | 5/3 | — | 1 | 2-3 | 1^{Ψ} | — | | 1-2 | — | 2-3 |
| A. fumigatus | 3/3 | — | 1-2 | 4 | 1^{Ψ} | — | 1-2 | 1^{Ψ} | — | 1^{Ψ} |
| S. cerevisiae | 6/3 | 1^{Ψ} | 3-4 | 3 | — | — | 2-4 | 2 | — | 1-2 |
| C6 glioma cells | 3/1 | — | 2-4 | 2-4 | 3-4 | — | 3-4 | — | — | — |
| ANIMAL STUDIES | | | | | | | | | | |
| Control brain | 19 | — | 3-4 | 2 | 3-4 | 3-4 | 2-3 | 1-2 | 2-3 | — |
| Glioma | 19 | — | 2 | 2 | 2 | 2 | 1-2 | — | — | — |
| Cryptococcoma | 20 | 3-4 | 1-2 | 2 | 1 | 1^{Ψ} | 3 | — | — | — |

Cross-peak volumes were compared with the most intense cross-peak in the respective COSY-spectrum. Signals were classified as 4 if their volume was 60-100%, 3 if their volume was 30-60%, 2 if their volume was 10-30% and as 1 if their volume was <10% of the most intense peak.
*Triglyceride peaks (32).
^{ψ}Not detected for all samples. Other abbreviations: ala, alanine; chol, choline; eth, ethanol; gaba, γ-amino butyric acid; glu/gln, glutamate/glutamine; GPC, glycero-phosphocholine; lys, lysine; inos, myo-inositol; NAA, N-acetyl aspartate; PC, phosphocholine; tau, taurine; tre, α,α-trehalose. Please note that detected amino acid residues do not necessarily represent free amino acids. Spectra from repeat cultures of some individual isolates have been included.

TABLE 6
Peak ratios for selected ¹H MR signals.

| | *No. of | | | | | |
|---|---|---|---|---|---|---|
| | samples/ | 3.25 ppm:3.05 ppm | 5.18 ppm:3.05 ppm | 5.38 ppm:3.05 ppm | 2.00 ppm:3.05 ppm | 5.38 ppm:5.18 ppm |
| Sample | isolates | mean ± SD (min-max) | mean ± SD (min-max) | mean ± SD (min-max) | mean ± SD (min-max) | mean ± SD (min-max) |
| CULTURES | | | | | | |
| C. neoformans | 24/16 | 2.1 ± 0.6 (1.4-3.0) | 4.7 ± 2.3 (0.5-8.0) | 1.5 ± 0.6 (0.6-2.3) | n.d. | 0.4 ± 0.3 (0.2-2.0) |
| C. albicans | 5/3 | 1.5 ± 0.3 (0.8-2.2) | 0.1 ± 0.1 (n.d.-0.1) | 0.9 ± 0.3 (0.4-1.9) | n.d. | 15 ± 7 (10-20) |
| A. fumigatus | 3/3 | 1.7 ± 0.4 (1.1-2.1) | n.d. | 0.4 ± 0.4 (n.d.-1.0) | n.d. | n.d. |
| S. cerevisiae | 6/3 | 1.4 ± 0.3 (1.0-1.9) | 0.1 ± 0.1 (n.d.-0.2) | 0.3 ± 0.2 (0.1-0.8) | n.d. | 7 ± 2 (5-10) |
| C6 glioma cell | 3/1 | 3.6 ± 1 (2.0-8.0) | n.d. | 0.5 ± 0.2 (n.d.-0.6) | n.d. | n.d. |
| line | | | | | | |
| ANIMAL | | | | | | |
| STUDIES | | | | | | |
| Control brain | 19 | 1.2 ± 0.3 (0.8-1.7) | n.d. | n.d. | 2.1 ± 0.4 (1.5-3.0) | n.d. |
| Cryptococcoma | 20 | 1.5 ± 0.3 (1.0-1.9) | 0.8 ± 0.4 (0.2-1.8) | 0.4 ± 0.2 (0.1-0.7) | 1.0 ± 0.7 (n.d.-2) | 0.6 ± 0.3 (0.3-1.2) |
| Glioma | 19 | 1.9 ± 0.4 (1.2-2.4) | n.d. | 1.3 ± 0.7 (0.2-3.0) | 0.9 ± 0.7 (n.d.-2) | n.d. |
| Ratios are expressed as mean values ± standard deviations (SD). The maximum and minimum value for the respective ratios are in brackets (min-max); n.d. - not detectable. Some signals are composite peaks. Main components of the designated signals are: 2.00 ppm - NAA and other acetyl groups; 3.05 ppm - creatine, phosphocreatine, lys residue, gaba and other compounds containing NCHₐ- groups; 3.25 ppm - choline, PC, GPC, betaine, tau, | | | | | | |
| #and other compounds containing N(CH₃)₃ groups; 5.18 ppm - H-1 of α,α-trehalose for all Cryptococcus neoformans cell and animal model samples or other anomeric protons of carbohydrate residues for in vitro cultured fungi other than Cryptococcus neoformans; 5.38 ppm - olefinic protons (CH═CH) of triglyceride acyl chains (for abbreviations, see legend to Table 1). | | | | | | |

*For some isolates n > 1 samples.

We claim:

1. A method for obtaining a statistical classifier for classifying microorganisms of unknown species into known species, comprising:
(a) obtaining a plurality of magnetic resonance spectra of each of a plurality of different species of microorganisms whose species is known;
(b) locating a plurality of maximally discriminatory subregions in the magnetic resonance spectra obtained; and
(c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, which coefficients and classifier spectra can be used to determine the species of microorganisms whose species are unknown.

2. The method of claim 1, further comprising the step of repeating step (c) a plurality of times, each time selecting as the first portion of the spectra a different portion of the spectra from the species, to obtain a different set of optimized linear discriminant analysis coefficients for the species, and obtaining a weighted average of the linear discriminant analysis coefficients to obtain final classifier spectra.

3. The method of claim 1, wherein the step of cross-validating the spectra comprises cross validating the spectra by randomly selecting about half of the spectra.

4. The method of claim 2, wherein the step of repeating step (c) a plurality of times comprises repeating step (c) about 1000 times.

5. The method of claim 1, further including the steps of obtaining a plurality of classifier spectra independently, and aggregating the results of the independent classifiers to obtain a consensus diagnosis.

6. The method of claim 1, wherein the microorganisms include bacteria.

7. The method of claim 6, wherein the bacteria includes the species of Staphylococcus aureus and Staphylococcus epidermidis.

8. The method of claim 6, wherein the bacteria includes the species of Enterococcus faecalis, Enterococcus casseliflavus and Enterococcus gallinarum.

9. The method of claim 6, wherein the bacteria includes the species of Streptococcus pneumoniae, Streptococcus pyogeries and Streptococcus agalachae.

10. The method of claim 1, wherein the microorganisms include fungi.

11. The method of claim 10, wherein the fungi includes pathogenic yeasts.

12. The method of claim 11, wherein the pathogenic yeasts include Candida albicans, Candida parapsilosis, Candida tropicalis, Candida krusei, and Candida glabrata.

13. The method of claim 11, wherein the pathogenic yeasts include Cryptococcus varieties.

14. The method of claim 13, wherein the Cryptococcus varieties include neoformans and gattli.

15. The method of claim 1, wherein the plurality of magnetic resonance spectra of each different species is at least 10.

16. The method of claim 1, wherein the plurality of magnetic resonance spectra of each different species is at least 30.

17. The method of claim 1, wherein the microorganisms include cultured bacterial infections.

18. The method of claim 1, wherein the microorganisms include specimens from a mammal containing bacterial infections.

19. A method for determining the species of a microorganism of unknown species, comprising:
Obtaining magnetic resonance spectra of the microorganism of unknown species, and comparing the spectra obtained with a species classifier, said classifier having been obtained by
(a) obtaining a plurality of magnetic resonance spectra of each of a plurality of different species of microorganisms whose species is known;
(b) locating a plurality of maximally discriminatory subregions in the magnetic resonance spectra obtained; and
(c) cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms; and selecting, as the species of the unknown species of microorganism, the microorganism whose spectra has the closest match to the spectra of the unknown microorganism species.

20. The method of claim 19, wherein the steps of obtaining the classifier further comprises the step of repeating step (c) a plurality of times, each time selecting as the first portion of the spectra a different portion of the spectra from the species, to obtain a different set of optimized linear discriminant analysis coefficients for the species, and obtaining a weighted average of the linear discriminant analysis coefficients to obtain final classifier spectra.

21. The method of claim 19, wherein the step of cross-validating the spectra comprises cross validating the spectra by randomly selecting about half of the spectra.

22. The method of claim 20, wherein the step of repeating step (c) a plurality of times comprises repeating step (c) about 1000 times.

23. The method of claim 19, further including the steps of obtaining a plurality of classifier spectra independently, and aggregating the results of the independent classifiers to obtain a consensus diagnosis.

24. The method of claim 19, wherein the microorganisms include bacteria.

25. The method of claim 24, wherein the bacteria includes the species of Staphylococcus aureus and Staphylococcus epidermidis.

26. The method of claim 24, wherein the bacteria includes the species of Enterococcus faecalis, Enterococcus casselifavus and Enterococcus gallinarum.

27. The method of claim 6, wherein the bacteria includes the species of Streptococcus pneumoniae, Streptococcus pyogeries and Streptococcus agalachae.

28. The method of claim 1, wherein the microorganisms include fungi.

29. The method of claim 28, wherein the fungi includes pathogenic yeasts.

30. The method of claim 29, wherein the pathogenic yeasts include Candida albicans, Candidaparapsilosis, Candida tropicalis, Candida krusei, and Candida glabrata.

31. The method of claim 29, wherein the pathogenic yeasts include Cryptococcus varieties.

32. The method of claim 31, wherein the Cryptococcus varieties include neoformans and gattli.

33. The method of claim 19, wherein the plurality of magnetic resonance spectra of each different species is at least 10.

34. The method of claim 19, wherein the plurality of magnetic resonance spectra of each different species is at least 30.

35. The method of claim 19, wherein the microorganisms include cultured bacterial infections.

36. The method of claim 19, wherein the microorganisms include specimens from a mammal containing bacterial infections.

37. A statistical classifier for classifying microorganisms of unknown species into known species, comprising:
(a) a spectrometer for obtaining a plurality of magnetic resonance spectra of each of a plurality of different species of microorganisms whose species is known;
(b) a locator for locating a plurality of maximally discriminatory subregions in the magnetic resonance spectra obtained; and
(c) a cross-validator for cross-validating the spectra by selecting a first portion of the spectra from each species, developing linear discriminant analysis classifiers from the first portion of the spectra from each species, and validating the remainder of the spectra from each species using the classifiers from the first portion of the spectra from each species to obtain optimized linear discriminant analysis coefficients and classifier spectra for each of the known species of microorganisms, which coefficients and classifier spectra can be used to determine the species of microorganisms whose species are unknown.

38. The classifier of claim 37, wherein the cross-validator repeats step (c) a plurality of times, each time selecting as the first portion of the spectra a different portion of the spectra from the species, to obtain a different set of optimized linear discriminant analysis coefficients for the species, and obtaining a weighted average of the linear discriminant analysis coefficients to obtain final classifier spectra.

39. The classifier of claim 37, wherein the cross-validator cross validates the spectra by randomly selecting about half of the spectra.

40. The classifier of claim 38, wherein the classifier repeats step (c) about 1000 times.

41. The classifier of claim 37, wherein the classifier obtains a plurality of classifier spectra independently, and aggregates the results of the independent classifiers to obtain a consensus diagnosis.

42. The classifier of claim 37, wherein the microorganisms includes bacteria.

43. The classifier of claim 42, wherein the bacteria includes the species of Staphylococcus aureus and Staphylococcus epidermidis.

44. The classifier of claim 42, wherein the bacteria includes the species of Enterococcus faecalis, Enterococcus casseliflavus and Enterococcus gallinarum.

45. The classifier of claim 42, wherein the bacteria includes the species of Streptococcus pneumoniae, Streptococcus pyogeries and Streptococcus agalachae.

46. The classifier of claim 37, wherein the microorganisms include fungi.

47. The classifier of claim 46, wherein the fungi includes pathogenic yeasts.

48. The classifier of claim 47, wherein the pathogenic yeasts include Candida albicans, Candida parapsilosis, Candida tropicalis, Candida krusei, and Candida glabrata.

49. The classifier of claim 47, wherein the pathogenic yeasts include Cryptococcus varieties.

50. The classifier of claim 49, wherein the Cryptococcus varieties include neoformans and gattli.

51. The classifier of claim 37, wherein the plurality of magnetic resonance spectra of each different species is at least 10.

52. The classifier of claim 37, wherein the plurality of magnetic resonance spectra of each different species is at least 30.

53. The classifier of claim 1, wherein the microorganisms include cultured bacterial infections.

54. The classifier of claim 1, wherein the microorganisms include specimens from a mammal containing bacterial infections.
